(19) United States
(12) Patent Application Publication
(10) Pub. No.: US 2026/0364573 A1
(43) Pub. Date: Nov. 19, 2026

(54) METHOD FOR PRODUCING BULK CATALYST SHAPED BODIES FOR GAS-PHASE OXIDATION OF AN ALKENE AND/OR AN ALCOHOL TO FORM AN A,B-UNSATURATED ALDEHYDE AND/OR AN A,B-UNSATURATED CARBOXYLIC ACID

(71) Applicant: BASF SE, Ludwigshafen am Rhein (DE)

(72) Inventors: Kazuhiko AMAKAWA, Ludwigshafen am Rhein (DE); Josef MACHT, Ludwigshafen am Rhein (DE); Christian WALSDORFF, Ludwigshafen am Rhein (DE); Markus MUEHL, Ludwigshafen am Rhein (DE); Cathrin Alexandra WELKER-NIEUWOUDT, Ludwigshafen am Rhein (DE); William Owen TUTTLE, Ludwigshafen am Rhein (DE); Thorsten JOHANN, Ludwigshafen am Rhein (DE); Samira PARISHAN, Ludwigshafen am Rhein (DE); David SCHLERETH, Ludwigshafen am Rhein (DE)

(21) Appl. No.: 19/103,250

(22) PCT Filed: Aug. 7, 2023
(86) PCT No.: PCT/EP2023/071775

§ 371 Date: Feb. 12, 2025

(30) Foreign Application Priority Data
Aug. 16, 2022 (EP) 22190557.3

(51) Int. Cl. B01J 23/887; B01J 35/30; B01J 35/31; B01J 35/55; B01J 35/63; B01J 37/00; B01J 37/08; C07C 45/35; C07C 51/235

(52) U.S. Cl. CPC B01J 23/8876; B01J 35/31; B01J 35/394; B01J 35/55; B01J 35/633; B01J 35/635; B01J 37/0036; B01J 37/0045; B01J 37/08; C07C 45/35; C07C 51/235

(57) ABSTRACT

The present invention relates to a process for producing shaped unsupported catalyst bodies for gas phase oxidation of an alkene and/or an alcohol to an α, β-unsaturated aldehyde and/or an α, β-unsaturated carboxylic acid, wherein a shaped precursor body having a density of 1.20 to 1.70 g/cm³ is produced by compaction, the shaped precursor body is subjected to thermal treatment, and the weight loss in the thermal treatment is from 25% to 45% by weight.

The present invention relates to a process for producing shaped unsupported catalyst bodies for gas phase oxidation of an alkene and/or an alcohol to an α, β-unsaturated aldehyde and/or an α, β-unsaturated carboxylic acid, wherein a shaped precursor body having a density of 1.20 to 1.70 g/cm³ is produced by compaction, the shaped precursor body is subjected to thermal treatment, and the weight loss in the thermal treatment is from 25% to 45% by weight.

The present invention also relates to the shaped unsupported catalyst bodies obtainable in accordance with the invention and to the use thereof for heterogeneously catalyzed partial gas phase oxidation over a fixed catalyst bed.

US 2005/0065371 describes shaped catalyst bodies comprising at least the elements molybdenum, bismuth and iron for gas phase oxidation.

US 2006/0036111 discloses a process for gas phase oxidation for production of an α, β-unsaturated aldehyde and/or an α, β-unsaturated carboxylic acid. Ignition loss in the course of calcination must be within a defined range here.

WO 2010/000720 teaches a process for producing shaped unsupported catalyst bodies.

WO 2013/007736 discloses shaped unsupported catalyst bodies comprising at least the elements molybdenum, bismuth, iron and cobalt for gas phase oxidation. The elements bismuth, iron and cobalt must be present here in a defined ratio.

WO 2015/067659 describes hollow cylindrical shaped catalyst bodies having a defined geometry. The shaped unsupported catalyst bodies have high stability and high product of value selectivity in the gas phase oxidation of propene to acrolein and acrylic acid.

WO 2021/239483 discloses shaped unsupported catalyst bodies having a defined cylindrical structure. The shaped unsupported catalyst bodies enable a high packing density and a low pressure drop in the shaped catalyst bed.

None of the above-cited patent applications ascribes any significance to the pressures expended in the production of the shaped precursor bodies or the density of the shaped precursor bodies.

It was an object of the present invention to provide an improved process for producing unsupported catalysts for gas phase oxidation of an alkene and/or an alcohol to give an α, β-unsaturated aldehyde and/or an α, β-unsaturated carboxylic acid. The catalysts were to have an improved product of value selectivity and an improved product of value yield. “Product of value” here means the sum total of acrolein and acrylic acid.

The object of the invention is achieved by a process for producing shaped unsupported catalyst bodies for gas phase oxidation of an alkene and/or an alcohol to an α, β-unsaturated aldehyde and/or an α, β-unsaturated carboxylic acid, where the shaped unsupported catalyst bodies comprise at least the elements molybdenum, bismuth, iron, cobalt and optionally nickel, where
a) at least one source of the elemental constituents molybdenum, bismuth, iron, cobalt and optionally at least one source of the elemental constituent nickel is used in each case to produce an aqueous solution or aqueous suspension,
b) a powder P is produced by drying the aqueous solution or aqueous suspension obtained in a) and optionally comminuting,
c) the powder P obtained in b), optionally with addition of one or more auxiliaries and after uniform mixing and optional compaction, is compacted to shaped precursor bodies having a cylindrical structure, and
d) the shaped precursor bodies obtained in c) are subjected to thermal treatment to form the shaped unsupported catalyst bodies,
wherein the pressure in the compaction in c) is chosen such that the density of the shaped precursor bodies is from 1.70 to 2.30 g/cm³, where the density of the shaped precursor bodies is the quotient of the mass and geometric volume, and the weight loss in the thermal treatment in d) is from 25% to 40% by weight.

The geometric volume is the macroscopic volume of the shaped precursor body including the pores. For the sake of completeness, it is emphasized that the essentially circular apertures and any surface structures such as grooves, notches or spikes are not part of the macroscopic volume.

A cylindrical structure is a cylindrical body, preferably having at least one circular aperture in the longitudinal direction. In the case of one aperture, this is arranged in the middle (hollow cylinder). In the case of multiple apertures, these are distributed homogeneously over the cross section of the cylindrical structure. A cylindrical structure having three apertures is described, for example, by WO 2021/239483.

The thermal treatment comprises an optional thermal pretreatment and the actual calcination.

The pressure in the compaction in c) is chosen such that the density of the shaped precursor bodies is preferably from 1.72 to 2.28 g/cm³, more preferably from 1.74 to 2.26 g/cm³, especially preferably from 1.76 to 2.24 g/cm³, very especially preferably from 1.78 to 2.22 g/cm³, most preferably from 1.80 to 2.20 g/cm³.

The weight loss in the thermal treatment in d) is preferably from 26% to 39% by weight, more preferably from 27% to 38% by weight, especially preferably from 28% to 37% by weight, very especially preferably from 29% to 36% by weight, most preferably from 30% to 35% by weight.

The weight loss in the thermal treatment may be adjusted, for example, via use of substances that break down in the course of thermal treatment.

Suitable cylindrical structures are cylindrical bodies having a central circular aperture in the longitudinal direction (hollow cylinders) and cylindrical bodies having three uniform notches in the longitudinal direction and having three circular apertures arranged uniformly in the longitudinal direction. The latter cylindrical structures are described in WO 2021/239483.

The shortest distance between the outer wall of the cylindrical body and the closest aperture is preferably from 0.75 to 2.5 mm, more preferably from 0.8 to 2.0 mm, especially preferably from 1.0 to 1.8 mm, very especially preferably from 1.2 to 1.7 mm, most preferably from 1.3 to 1.6 mm. In the case of a hollow cylinder, the shortest distance between the outer wall of the cylindrical body and the closest aperture corresponds to the wall thickness of the hollow cylinder.

The molybdenum content of the shaped unsupported catalyst bodies, calculated as MoO₃, preferably from 45% to 75% by weight, more preferably from 50% to 70% by weight, most preferably from 55% to 65% by weight.

The bismuth content of the shaped unsupported catalyst bodies, calculated as Bi₂O₃, from 1% to 20% by weight, more preferably from 2% to 15% by weight, most preferably from 3% to 10% by weight.

The iron content of the shaped unsupported catalyst bodies, calculated as Fe₂O₃, from 2% to 12% by weight, more preferably from 3% to 11% by weight, most preferably from 4% to 10% by weight.

The total cobalt and nickel content of the shaped unsupported catalyst bodies, calculated as CoO and NiO, from 9% to 30% by weight, more preferably from 12% to 27% by weight, most preferably from 15% to 24% by weight.

The shaped unsupported catalyst bodies may additionally comprise the elements potassium and/or silicon.

The present invention further provides the shaped unsupported catalyst bodies having a cylindrical structure that are produced by the process of the invention for gas phase oxidation of an alkene and/or an alcohol to an α, β-unsaturated aldehyde and/or an α, β-unsaturated carboxylic acid, where the shaped unsupported catalyst body comprises at least the elements molybdenum, bismuth, iron and cobalt, wherein the density of the shaped unsupported catalyst body is from 1.20 to 1.70 g/cm³, where the density of the shaped unsupported catalyst body is the quotient of the mass and geometric volume, the total pore volume of the shaped unsupported catalyst body is from 0.33 to 0.60 cm³/g and the pore volume of the shaped unsupported catalyst body in the range from 0.1 to 1 μm is from 85% to 99% of the total pore volume, where the total pore volume and the pore volume in the range from 0.1 to 1 μm is determined by mercury porosimetry.

The geometric volume is the macroscopic volume of the shaped unsupported catalyst body including the pores. For the sake of completeness, it is emphasized that the essentially circular apertures and any surface structures such as grooves, notches or spikes are not part of the macroscopic volume.

The density of the shaped unsupported catalyst body is preferably from 1.22 to 1.68 g/cm³, more preferably from 1.24 to 1.66 g/cm³, especially preferably from 1.26 to 1.64 g/cm³, very especially preferably from 1.28 to 1.62 g/cm³, most preferably from 1.30 to 1.60 g/cm³.

The total pore volume of the shaped unsupported catalyst body is preferably from 0.34 to 0.58 cm³/g, more preferably from 0.35 to 0.56 cm³/g, especially preferably from 0.36 to 0.54 cm³/g, very especially preferably from 0.37 to 0.52 cm³/g, most preferably from 0.38 to 0.50 cm³/g.

The pore volume of the shaped unsupported catalyst body in the range from 0.1 to 1 μm is preferably from 86% to 98%, more preferably from 87% to 97%, especially preferably from 88% to 96%, most preferably from 89% to 95%, of the total pore volume.

Preferred shaped unsupported catalyst bodies are multielement oxides of the general formula I

Mo₁₂BiₐFe_{b}Co_{c}Ni_{d}XₑYₜZ_{g}Oₙ (1)
with
X=K, Cs and/or Rb
Y=Ca, Sr, Ba, Li, Na, Cr, W, Mn, Cu, Zn, Ga, P, B, As, Sn, Sb, Te, Nb, Ta, Pb, Ce and/or La
Z=Si, Al, Ti, Zr and/or Mg
a=0.2 to 2
b=1 to 4
c=3 to 9
d=0 to 4
c+d=4 to 9.5
e=0.01 to 0.5
f=0 to 10
g=0 to 10
n=a number which is determined by the valency and frequency of the elements in the general formula I other than oxygen.

The present invention further provides processes for preparing an α, β-unsaturated aldehyde and/or an α, β-unsaturated carboxylic acid, wherein an alkene and/or an alcohol, especially propene, is passed together with molecular oxygen over a fixed catalyst bed comprising a bed of shaped unsupported catalyst bodies of the invention, and fixed bed reactors comprising a bed of shaped unsupported catalyst bodies of the invention.

The present invention is based on the finding that the pressure expended in the production of the shaped precursor bodies and the weight loss that occurs in the thermal treatment have a significant effect on the product of value selectivity and the product of value yield.

The production of the shaped unsupported catalyst bodies is described below.

The shaped unsupported catalyst bodies obtainable in accordance with the invention are typically formed in substance to geometric shaped bodies, calcined, and used for catalysis of the respective heterogeneously catalyzed gas phase partial oxidation (especially that of propene to acrolein). In principle, the desired geometry of the unsupported catalysts is not subject to any restriction.

In principle, shaped unsupported catalyst bodies can be produced in a simple manner by using suitable sources of their elemental constituents (especially other than oxygen) to produce a very intimate, preferably finely divided, dry mixture of corresponding composition to the respective stoichiometry of the shaped unsupported catalyst bodies to be produced, and, after previous shaping to shaped precursor bodies, which is optionally effected with additional use of shaping auxiliaries, calcining them at temperatures of 350 to 650 °C. The calcination can be effected either under inert gas or under an oxidative atmosphere, for example air (or another mixture of inert gas and molecular oxygen that may also comprise reducing constituents in comparatively small proportions) or else under a reducing atmosphere (for example a mixture of inert gas, NH₃, CO and/or H₂ that may also comprise oxidizing constituents in comparatively small proportions), or under reduced pressure. The calcination time may be a few minutes to a few days and is typically shorter at elevated calcination temperature.

Useful sources for the elemental constituents of the shaped unsupported catalyst bodies (i.e. as starting compounds that comprise at least one elemental constituent (at least one element present in the shaped unsupported catalyst body) in chemically bound form) include those compounds that are already oxides (generally in the solid state of matter under standard conditions (1 atm, 0 °C)) (for example metal oxides) and/or those compounds that are convertible to oxides (which are generally in the solid state of matter under standard conditions) by heating (thermal treatment at elevated temperature) at least in the presence of gaseous oxygen and/or of components that release gaseous (for example molecular) oxygen. In principle, the oxygen source may, for example, be in the form of a peroxide constituent of the mixture to be calcined. It is quite generally the case that one starting compound may be the source for more than one elemental constituent of the shaped unsupported catalyst bodies.

As well as the oxides, useful starting compounds (sources) of this kind include, in particular, halides, nitrates, formates, acetates, oxalates, citrates, carbonates, ammine complexes, ammonium salts and/or hydroxides and hydrates of the aforementioned salts.

Compounds such as NH₄OH, (NH₄)₂CO₃, NH₄NO₃, NH₄CHO₂, CH₃COOH, NH₄CH₃CO₂ and/or ammonium oxalate that break down and/or decompose in the later calcining or earlier essentially completely to give compounds that escape in gaseous form (for example ammonia, CO₂, CO, H₂O, nitrogen oxides) can additionally be incorporated into the intimate dry mixture. Useful substances of this kind that break down in the calcining operation also include organic materials, for example stearic acid, malonic acid, ammonium salts of the aforementioned acids, starches (for example potato starch, corn starch), ground nutshells and finely divided ground polymer (for example polyethylene, polypropylene etc.).

There is normally also formation (release) of gaseous compounds in the course of the thermal treatment outlined when the element sources with which the very intimate and preferably finely divided dry mixture is produced are partly organic in nature (for example in the case of acetates, formates, oxalates and/or citrates) or comprise hydroxide ions, carbonate ions, hydrogencarbonate ions, ammonium ions, halide ions, hydrogenphosphate ions and/or nitrate ions that normally break down in the calcining operation.

The intimate mixing of the starting compounds (sources) for production of shaped unsupported catalyst bodies can be effected in dry or wet form. If it is effected in dry form, the starting compounds (the sources) are appropriately used in the form of fine powders and, after the mixing and compaction to give the geometric shaped precursor body, subjected to the calcination.

Preferably in accordance with the invention, however, the intimate mixing of the (element) sources is effected in wet form.

In this case, the starting compounds in the form of solutions and/or suspensions are mixed with one another, and the resulting wet (preferably aqueous) mixture M is then dried to give the intimate dry mixture. The solvent and/or suspension medium used is preferably water or an aqueous solution, resulting in an aqueous mixture M as wet mixture M.

Very particularly intimate dry mixtures are obtained in the mixing process described above when the starting materials are exclusively sources of the elemental constituents in dissolved form and/or in colloidally dissolved form. As already mentioned, a starting compound may be a source for just one or more than one elemental constituent. In a corresponding manner, an above-detailed solution or colloidal solution may include just one or else more than one elemental constituent of the relevant shaped unsupported catalyst body to be produced in dissolved form. The preferred solvent here, as already stated, is water. The drying of the resulting aqueous mixtures is preferably effected by spray drying.

If the present document refers to a solution of a source (starting compound, starting substance) in a solvent (especially water), the term “dissolving” is meant in the sense of a molecular or ionic solution. This means that the largest geometric unit of the dissolved starting substance (source) present in the solution inevitably has “molecular” dimensions, and the solution appears “visually empty”.

By contrast, colloidal solutions constitute a link between true (molecular and/or ionic) solutions and suspensions. In these colloidally dispersed systems, there are smaller accumulations of molecules or atoms, but these are not apparent either to the naked eye or by microscope. The colloidal solution has an entirely clear (although often colored) visual appearance, since the particles present therein have only a diameter of 1 to 250 nm (preferably to 150 nm and more preferably to 100 nm).

Owing to their small size, it is not possible to remove the colloidally dissolved particles by conventional filtration. However, they can be separated from their “solvent” by ultrafiltration with membranes of vegetable, animal or synthetic origin (for example parchment, pig's bladder or cellophane). By contrast with the “visually empty” true (molecular and/or ionic) solutions, a light beam cannot pass through a colloidal solution without deflection. The light beam is scattered and deflected by the colloidally dissolved particles. In order to keep colloidal solutions stable and to prevent further particle agglomeration, they frequently comprise added wetting aids and dispersing aids and other additives.

While elements (elemental constituents) other than silicon for a shaped unsupported catalyst body are preferably introduced by dissolved sources in the form of a solution (more preferably in an aqueous solution) for production of the wet (preferably aqueous) mixture M, the element silicon is preferably introduced in the form of a silica sol for production of the wet (preferably aqueous) mixture M.

Silica sols are aqueous colloidal solutions of virtually spherical polysilicic acid particles. The diameter of the particles is in the colloidal range and, according to type, is 5 to 75 nm. The particles are pore-free. They have a core of SiO₂ that has been hydroxylated on its surface.

The spherical individual particles are not crosslinked to one another. For reasons of stability, in silica sols, a portion of the hydroxyl groups is normally in alkali metal hydroxide- and/or ammonium hydroxide-neutralized form. This means that some of the counterions in that case are not protons, but alkali metal ions (for example Na⁺) and/or NH₄⁺ cations. The SiO₂ content of silica sols suitable for production of a wet (preferably aqueous) mixture M may, for example, be 30% to 60% of the weight of the silica sol. Silica sols are normally water-fluid and do not comprise any sedimentable constituents. They can often be kept for years without sedimentation.

Particularly suitable Si sources are the LUDOX* silica sols from Grace GmbH & KG, In der Hollerecke 1, D-67545 Worms. The particles thereof are discrete spherical spheres of silicon dioxide with no internal surface area or detectable crystallinity. The predominant proportion thereof is dispersed in an alkaline medium that reacts with the hydroxylated surface and results in repellent negative charges.

The particle diameters of suitable silica sols may be very narrow (essentially monodisperse) or very broad (polydisperse).

A silica sol particularly suitable for the purposes of the invention (for production of a wet (preferably aqueous) mixture M) is the LUDOX TM50 silica sol from Grace. The LUDOX TM50 silica sol has a very substantially monodisperse (d=22 nm) particle diameter distribution. Its pH (1 atm, 25 °C) is 9.0. The alkali metal ion that replaces the portion of the hydroxyl protons is Na⁺. The SiO₂ content of LUDOX TM50 is 50% of the weight of this hydrogel. The specific surface area of the SiO₂ particles present in colloidally dissolved form in LUDOX TM50 is 140 m²/g. The bulk density (1 atm, 25 °C) of LUDOX TM50 is 1.40 g/cm³. The titratable alkali metal content of LUDOX TM50 (calculated as Na₂O) is 0.21% by weight (based on the weight of the silica sol).

The dynamic viscosity of LUDOX TM50 is 40 mPas (1 atm, 25 °C). The content of Cl⁻ (calculated as NaCl) of LUDOX TM50 is 0.03% by weight and the content of SO₄²⁻ of LUDOX TM50 (calculated as Na₂SO₄) is 0.08% by weight (based in each case on the weight of LUDOX TM50).

It will be appreciated that it is also possible for at least one element source in molecularly and/or ionically dissolved form and one or more than one other element source in colloidally dissolved form to be present alongside one another in a solution to be used for production of a wet (especially aqueous) mixture M.

A favorable Mo source is ammonium heptamolybdate tetrahydrate. This is particularly because it has excellent solubility in water. According to Ullmann's Encyclopedia of Industrial Chemistry, Volume 22, 2003, WILEY-VCH, pages 320/321, a solution of ammonium molybdate tetrahydrate in water at 25 °C and 1 atm has a saturation solubility of 30% by weight (calculated as anhydrous salt).

As a result of production, ammonium molybdate tetrahydrate (as a consequence of process parameters not accurately observed in the production thereof) may be contaminated in minor amounts (usually ppm) with water-insoluble isopolymolybdate. If ammonium heptamolybdate tetrahydrate contaminated in this way is dissolved in water, this gives rise to an aqueous solution that has a certain turbidity owing to the small amounts of finely divided isopolymolybdate present in undissolved form. In-house studies by the applicant have shown that even isopolymolybdate-contaminated ammonium molybdate tetrahydrate, the solution of which in water (determined as described in WO 2016/147324) has a turbidity of 20 NTU, or of 50 NTU, or of 70 NTU, or of 100 NTU, or of 150 NTU, or of 200 NTU, or of 250 NTU, or 300 NTU, is suitable for production of inventive shaped unsupported catalyst bodies without any perceptible impairment of the performance thereof when used as catalysts for the heterogeneously catalyzed partial gas phase oxidation of propene to acrolein as main product and acrolein as by-product.

Further suitable Mo sources are, for example, ammonium orthomolybdate ((NH₄)₂MoO₄), ammonium dimolybdate ((NH₄)₂Mo₂Oy), ammonium tetramolybdate dihydrate ((NH₄)₂Mo₄O₁₃×2 H₂O) and ammonium decamolybdate dihydrate ((NH₄)₄Mo₁₀O₃₂×2 H₂O). In principle, molybdenum trioxide is also usable.

A preferred source for alkali metals in the context of production of shaped unsupported catalyst bodies is the hydroxides thereof. In principle, the nitrates of these elements and the hydrates of these nitrates are also useful as such sources. This means that a preferred K source is KOH, but KNO₃ or the hydrate thereof can also be used in principle as the K source.

Bi sources used are preferably salts of bismuth that have Bi in the form of Bi³⁺. Examples of useful salts of this kind include bismuth(III) oxide, bismuth(III) oxide nitrate (bismuth subnitrate), bismuth(III) halide (for example fluoride, chloride, bromide, iodide) and especially bismuth(III) nitrate pentahydrate. It will be appreciated that the Bi source used may also be a solution of elemental Bi in aqueous nitric acid in which the Bi is in the form of Bi³⁺. In the case of the use (preferred in accordance with the invention) of an aqueous solution of Bi³⁺ nitrate or the hydrate thereof as source (such a solution can also be produced by dissolving elemental Bi in aqueous nitric acid), it is advantageous in accordance with the invention when the pH thereof (1 atm, 25 °C) is low since this counteracts unwanted formation of Bi³⁺-comprising precipitates in the aqueous solution. This pH is preferably ≤1, more preferably ≤0.5. In general, this pH, however, is ≥−2, usually ≥0. Appropriately, such an aqueous solution is a nitric acid solution. This means that its low pH is brought about by excess nitric acid (in this case, the molar ratio (n_{NO3-})/(n_{Bi3+}) of the molar amount of NO₃⁻ (n_{N03-}) present in the aqueous solution to the molar amount of Bi₃⁺ (n_{Bi3}) present in the aqueous solution is >3).

Preferred Fe sources are salts of Fe³⁺, among which particular preference is given to the various iron(III) nitrate hydrates (see, for example, DE 10 2007 003076 A1). Particular preference is given in accordance with the invention to using iron(III) nitrate nonahydrate as Fe source for the aforementioned purpose. It is of course also possible to use salts of Fe²⁺ as Fe source.

Advantageously, for production of the shaped unsupported catalyst bodies, based on the total molar amount of Fe present therein, at least 50 mol %, better at least 75 mol % and preferably at least 95 mol % or 100 mol % is introduced in the form of an Fe source having the Fe in the form of Fe³⁺. It is also possible for this purpose to use Fe sources having both Fe³⁺ and Fe³⁺.

Co sources of good suitability are especially the salts thereof that have the Co in the form of Co²⁺ and/or Co³⁺. Examples of these include cobalt(II) nitrate hexahydrate, Co₃O₄, COO, cobalt(II) formate and cobalt(III) nitrate. For the aforementioned purpose, particular preference is given to the first of these sources. It will be appreciated that the Co source used may also be a solution of elemental Co in aqueous nitric acid in which the Co is in the form of Co²⁺.

In the case of the elemental constituent Ni, preference is given to using Ni²⁺ salts. These especially include nickel(II) carbonate, nickel(II) sulfate, nickel(II) oxide, nickel(II) acetate, nickel(II) formate, nickel(II) hydroxide, nickel(II) oxalate and nickel(II) nitrate and the respective hydrates of these salts. Very particular preference is given to using hydrates of nickel(II) nitrate (for example the hexahydrate thereof) as Ni sources.

The solubility of, for example, salts of Fe, Co, Cu and/or Ni in an aqueous medium, can be improved if required by adding ammonia (or else the aqueous solution thereof) and/or nitric acid (especially as the aqueous solution thereof) to the respective solution.

In principle, the production of a wet (for example aqueous) mixture M can be effected under a wide variety of different gas atmospheres (for example under air, argon, nitrogen, steam and/or carbon dioxide). Preferably in accordance with the invention, the production of a wet (for example aqueous) mixture M is effected under air (advantageously, the aqueous mixture M is saturated in air). This is especially true when the cobalt and ion sources used are salts of Co²⁺ and salts of Fe²⁺. This is particularly true when these salts are nitrates and/or hydrates thereof.

As already mentioned, the wet mixture M, preferably in accordance with the invention, is an aqueous mixture M which is particularly advantageously produced in the manner which follows.

At least one source of the element Fe, at least one source of the element Bi, at least one source of the element Co and optionally at least one source of the element Ni are used to produce an aqueous solution A having a pH of ≤3, preferably ≤2, more preferably ≤1 and most preferably ≤0 (pH values of aqueous solutions relate generally in this document (unless explicitly stated otherwise) to a measurement with a glass electrode in the form of a combination electrode at 1 atm and at the temperature at which the respective aqueous solution is produced; the calibration of the combination electrode required for this purpose is effected under the same conditions by means of aqueous buffer solutions that have a known pH under these conditions which is close to the measurement sought; the Mettler Toledo Inpro 4260/425/Pt 100 pH electrode is especially suitable for determining such pH values, this being a combination electrode with integrated Pt 100 temperature sensor for automatic temperature compensation). In general, the pH of the aqueous solution A is not less than −2 and is particularly advantageously in the range of −1 to 0. Preferably, the aqueous solution A is an aqueous solution of the nitrates or nitrate hydrates of the aforementioned elements. More preferably, the aqueous solution A is an aqueous solution of these nitrates or nitrate hydrates in aqueous nitric acid. Particularly for production of such a solution, suitable element sources are also solutions of the relevant elements in aqueous nitric acid.

At least one source of the element Mo and optionally at least one of the sources of an alkali metal are used to produce an aqueous solution B. The pH of the aqueous solution B (at 1 atm and that temperature at which solution B is produced) is advantageously <7. More preferably, the pH of the aqueous solution B is ≤6.5 and very particularly advantageously ≤6. In general, the pH of the aqueous solution B will be ≥3. Favorable solutions B for use in accordance with the invention have a pH of 4 to 6. Preferably in accordance with the invention, for preparation of the aqueous solution B, the source used for an alkali metal is the element hydroxide thereof (for example KOH). A preferred Mo source for preparation of an aqueous solution B is ammonium heptamolybdate tetrahydrate ((NH₄)₆Mo₇O₂₄×4 H₂O), which is entirely soluble in water at 25 °C (1 atm) up to saturation solubility (30% by weight, calculated in anhydrous form).

Appropriately in accordance with the invention, the total content in the aqueous solution A of the metal constituents Bi, Fe, Co, Ni, etc., based on the total content of the aqueous solution A, is 5% to 20% by weight, advantageously 10% to 15% by weight.

Appropriately in accordance with the invention, the total content in the aqueous solution B of Mo, based on the total amount of the aqueous solution B, is 2% to 25% by weight, advantageously 3% to 20% by weight and particularly advantageously 5% to 15% by weight.

Subsequently, the aqueous solution A and the aqueous solution B are appropriately mixed with one another. The procedure here is advantageously to stir the aqueous solution A continuously into the aqueous solution B. This is advantageously done while vigorously stirring the initially charged aqueous solution B.

Appropriately in accordance with the invention, the total content in the resulting aqueous mixture of aqueous solution A and aqueous solution B of the metal constituents Bi, Fe, Co, Ni, Mo etc., based on the total content of the aqueous mixture, is 3% to 20% by weight, advantageously 5% to 15% by weight.

The temperature of the initially charged aqueous solution B and on stirring of the aqueous solution A into it, just like the temperature of the aqueous solution A itself, is advantageously (preferably over the entire mixing operation) ≤80 °C and ≥0 °C. Preferably, the aforementioned temperatures are ≤75 °C and ≥30 °C, and they are more preferably ≤70 °C and ≥50 °C or ≤65 °C and ≥55 °C.

Advantageously, the aqueous solutions A and B and the aqueous mixture that results on stirring of the aqueous solution A into the aqueous solution B have the same temperature. This temperature is at best 60 °C. Preferably, the temperatures of the aqueous solution A, the aqueous solution B and the aqueous mixtures that result from these are kept constant over the course of the stirring-in process described. For this purpose, for example, thermostat control is possible with the aid of a water bath. The working pressure on stirring of the aqueous solution A into the aqueous solution B is appropriately 1 atm (1.01 bar).

Preferably, the aqueous solution A is stirred into the initially charged aqueous solution B within a period within the range from 5 to 60 minutes, more preferably within a period of 10 to 30 minutes and most preferably within a period of 15 to 25 minutes. The resulting aqueous mixture is subsequently, preferably while maintaining the stirring-in temperature, stirred appropriately for a further 5 to 60 minutes, preferably 10 to 30 minutes and particularly advantageously 15 to 25 minutes.

The pH of the aqueous mixture of the aqueous solution A and the aqueous solution B is advantageously ≤3, better ≤2. In general, it is at values of ≥0.

If the shaped unsupported catalyst body comprises the elemental constituent Si, preferably in accordance with the invention, aqueous silica sol as the source thereof is stirred into the aqueous mixture of aqueous solution A and aqueous solution B, in which case water may advantageously have been added to this aqueous mixture prior to this stirring-in. In an appropriate manner, both the aqueous silica sol and the water may be added all at once. Both the temperature of the water and the temperature of the aqueous silica sol corresponds advantageously to the temperature of the aqueous mixture of the aqueous solution A and the aqueous solution B. Finally, in an appropriate manner, stirring is continued for up to 30 minutes. During the further stirring, the aforementioned temperature is advantageously maintained. The SiO₂ content of the added aqueous silica sol may be 15% to 60% by weight, or 20% to 60% by weight, or 30% to 60% by weight, preferably 40% to 60% by weight and more preferably 45% by weight to 55% by weight (based in each case on the total weight thereof).

Rather than initially charging the aqueous solution B in a thermostatted stirred vessel and then feeding the aqueous solution A into it while stirring, it is also possible to supply both the aqueous solution B and the aqueous solution A continuously to the stirred vessel (for example by means of a “3-way T mixer”). In principle, the aqueous solution B can also be stirred continuously into an initially charged aqueous solution A. However, this procedure is less preferred.

In general, the aqueous mixture M obtainable as described is an aqueous suspension (preferably, the ratios V described as advantageous likewise exist in the aqueous mixture M (total molar amount of NH₃+NH₄⁺ present to molar amount of Mo present); in addition, the pH of the aqueous mixture M obtainable as described is also advantageously ≤3, generally 0 to 2). Aqueous mixtures M obtainable as described advantageously comprise not more or less than 60 mol % of the total molar amount of Co and/or Ni present therein in a form dissolved in the aqueous medium (at that temperature and the working pressure at which the aqueous mixture M was produced). Preferably, the above-detailed proportion AT of the molar amount of Co and/or Ni present in the aqueous mixture M which is dissolved in the aqueous medium of the aqueous mixture M is at values of ≤50 mol % and more preferably at values of ≤40 mol %, or ≤30 mol % or ≤20 mol %. Appropriately in accordance with the invention, the total content in the aqueous mixture M to be dried (preferably spray-dried) of Bi, Fe, Mo etc., based on the amount of the aqueous mixture M, is 3% to 20% by weight, advantageously 5% to 15% by weight. In general, AT is ≥10 mol %, or ≤15 mol %.

Preferably in accordance with the invention, the aqueous mixture M is converted to a finely divided intimate dry mixture by spray-drying the aqueous mixture M (the aqueous mixture M is preferably dried as soon as possible after its production). This means that the aqueous mixture M is first divided (sprayed) into fine droplets in a spray drier and subsequently dried in the spray drier. The spray drying is preferably effected in a hot air stream. In principle, for the aforementioned spray drying, it is alternatively possible to use other hot gases (for example nitrogen or nitrogen-diluted air and other inert gases).

The spray drying can be effected either in cocurrent or in countercurrent of the droplets relative to the hot gas. Typical gas inlet temperatures are in the range here from 250 to 450 °C, preferably 270 to 370 °C. Typical gas outlet temperatures are in the range here from 100 to 160 °C. The spray drying is preferably effected in cocurrent flow of the droplets relative to the hot gas.

The average particle diameter of the resulting spray powder is typically 10 to 100 μm, preferably 15 to 60 μm and more preferably 25 to 50 μm (the diameter is determined to ISO 13320-1 by means of light scattering on air-dispersed spray powder (dispersion air pressure 1.0 bar). In general, references to a standard in this document relate in each case to that version of the standard that was in force at the priority date of this patent application and has a release date (issue date) that has the smallest time difference from the priority date of this patent application.

The tapped bulk density (25 °C, 1 atm) of the spray powder is typically 500 to 1300 g/L and preferably 700 to 1100 g/L.

The ignition loss of the spray powder (calcine at 600 °C (powder temperature) under an excess of stationary air for 3 h) is typically 20% to 40% by weight, preferably 25% to 35% by weight, of its starting weight.

Until further processing thereof, the spray powder can be intermediately stored in preferably airtight containers (for example drums manufactured from plastic). The storage temperature should not exceed 70 °C and is preferably ≤50 °C. In general, the storage temperature will not go below 10 °C. Since the spray powder is generally hygroscopic, extended contact thereof with moist air should be avoided. Contact with moist air can impair the handling properties of the spray powder (for example the flowability thereof) and ultimately reduce the catalytic activity of the shaped unsupported catalyst bodies produced therewith.

It will be appreciated that the aqueous mixture M can also be dried by other drying methods, for example conventional evaporation (preferably under reduced pressure; the drying temperature will generally not exceed 150 °C). In principle, the drying of an aqueous mixture M can also be effected by freeze drying or Spin-Flash® drying.

The spray powder first be coarsened, for example, by subsequent compaction. If the compaction is effected in dry form, prior to the compaction, finely divided graphite and/or other shaping auxiliaries mentioned in this document (for example lubricants, reinforcers and/or pore formers), for example, can be mixed into the spray powder (for example with a drum hoop mixer). For example, the compaction can be conducted with a calender having two counter-rotatory steel rolls. Subsequently, the compactate can be comminuted specifically to the particle size appropriate for the further use contemplated. The simplest way of doing this is, for example, to force the compactate through a sieve with defined mesh size.

In principle, the compaction can alternatively be effected in moist form. For example, the spray powder can be kneaded with addition of water. After the kneading, the kneaded composition can be comminuted back to the desired fineness matched to the subsequent use (cf., for example, DE 100 49 873 A1) and dried.

The finely divided precursor composition (the finely divided intimate dry mixture of the sources of the elemental constituents) is appropriately used to form shaped precursor bodies of regular or irregular geometry by compaction (compressing or compacting), and they are subsequently converted to the shaped unsupported catalyst bodies by thermal treatment.

Further finely divided shaping auxiliaries that may be added to the finely divided precursor composition before and/or during the shaping are, for example, again lubricants, for example graphite, carbon black, polyethylene glycol, polyacrylic acid, stearic acid, starch, mineral oil, vegetable oil, water, boron trifluoride and/or boron nitride. Also useful as shaping auxiliaries are reinforcers such as microfibers of glass, asbestos, silicon carbide or potassium titanate, which, after the shaping by compaction has ended, have a beneficial effect on the integrity of the compact obtained (of the resulting shaped body). Other useful shaping auxiliaries are pore formers such as ammonium nitrate, ammonium carbonate, water and/or malonic acid. The pore formers break down or evaporate forming pores during the thermal treatment. Additional use of lubricants in the context of a corresponding shaping operation is described, for example, in documents DE 10 2007 004961 A1, WO 2008/087116, WO 2005/030393, US 2005/0131253, WO 2007/017431, DE 10 2007 005606 A1 and DE 10 2008 040093 A1.

Preference is given to using exclusively finely divided graphite as lubricant. Finely divided graphites that are to be used are especially those which are recommended in WO 2005/030393, US 2005/0131253, WO 2008/087116 and DE 10 2007 005606 A1. This is especially true of those graphites that are used in the examples and comparative examples in these documents. Very particularly preferred graphites are Asbury 3160 and Asbury 4012 from Asbury Graphite Mills, Inc., New Jersey 08802, USA, and Timrex® T44 from Timcal Ltd., 6743 Bodio, Switzerland.

Based on the weight of the finely divided precursor composition to be shaped, it may comprise, based on its total weight, for example, up to 15% by weight of finely divided lubricant (for example graphite). Usually, however, the lubricant content in the finely divided precursor composition to be shaped (in the finely divided intimate dry mixture) is ≤9% by weight, in many cases ≤5% by weight, often ≤4% by weight; this is especially the case when the finely divided lubricant is graphite. In general, the aforementioned added amount is ≥0.5% by weight, usually ≥2.5% by weight.

In general, the finely divided precursor composition optionally comprising shaping auxiliaries (the finely divided intimate dry mixture) is compacted to the desired geometry of the shaped precursor body by the action of external forces (pressure) on the precursor composition. The shaping apparatus to be employed here, or the shaping method to be employed here, is not subject to any restriction.

For example, the compacting shaping can be effected by tableting. The finely divided precursor composition (the finely divided intimate dry mixture) used here is preferably dry to the touch. However, it may comprise, for example, up to 10% of its total weight of added substances that are liquid under standard conditions (25 °C, 1 atm (1.01 bar)). The finely divided precursor composition (the finely divided intimate dry mixture) may also comprise solid solvates (e.g. hydrates) that include such liquid substances in chemically and/or physically bound form. It will be appreciated that the finely divided precursor composition may also be entirely free of such substances.

A preferred shaping process by compaction of the finely divided precursor composition (of the finely divided intimate dry mixture) is tableting. The principles of tableting are described, for example, in “Die Tablette”, Handbuch der Entwicklung, Herstellung und Qualitstssicherung [“The Tablet”, Handbook of Development, Production and Quality Assurance], W. A. Ritschel and A. Bauer-Brandl, 2nd edition, Edition Verlag Aulendorf, 2002, and are applicable in an entirely corresponding manner to a tableting process of the invention.

Tableting is a press agglomeration process. The free-flowing feed mixture is introduced into a press mold having a die between two punches and compacted by monoaxial compression and shaped to a solid compacted body. Tableting can be divided into four parts: metered introduction, compaction (elastic deformation), plastic deformation and ejection. Tableting is effected, for example, on rotary presses or eccentric presses.

The outer surface of the tableted catalyst support consists of a circumferential surface corresponding to the inner wall of the die cavity, and of a first end lateral face and a second lateral face that correspond to the operative heads of the punches. The tableted catalyst support may be flat or have curved ends, meaning that at least one of the first lateral faces and the second lateral face are curved. Curved lateral faces may be obtained, for example, by use of a concave lower and/or upper punch. If desired, the upper punch and/or the lower punch may comprise projecting pins in order to form internal passages. It is also possible to provide the press punch with a multitude of pins, such that a punch can be produced, for example, with four pins in order to produce shaped bodies having four holes (passages). Typical construction features of such press molds can be found, for example, in U.S. Pat. No. 8,865,614.

Press molds typically consists of a die, an upper punch, a lower punch and pins (if the formed body has passages). Suitable materials for press molds are tool steels, cemented carbides based on tungsten carbide (WC) and ceramic materials. Tool materials having a hardness of more than 55 on the Rockwell C scale are preferred. Examples of tool steel materials are DIN tool steels 1.2210, 1.2343, 1.2436, 1.2379, 1.2601, 1.2080, 1.25550 and high-speed steels, Vanadis 4 Extra from Uddeholm D-40549 Dusseldorf, Vanadis 8 from Uddeholm D-40549 Dusseldorf. Suitable WC-based materials are described in U.S. Pat. No. 8,865,614. Examples of such WC-based materials are G10-Ni from Hartmetall® Gesellschaft in D70497 Stuttgart and htc-KR17® from Hightech-Ceram®. Examples of ceramic materials are yttrium-stabilized zirconia (YSZ).

WC-based cemented carbides and ceramic materials are particularly suitable for tool use, where a lined die made of WC-based cemented carbide or ceramic is inserted into a steel housing made of tool steel, e.g. 1.2379.

The press mold typically has a surface coating for improvement of surface hardness, corrosion resistance, wear resistance, friction and nonstick properties. Examples of surface coating types are diamond such as carbon (DLC), boron nitride, titanium nitride, chromium nitride, chrome plasma coating, hard chrome-plating. The thickness of the layer is 1 to 10 μm, preferably 1 to 5 μm.

The surface of press molds that come into contact with the feed mixture and the resulting tablets preferably have low surface roughness. The arithmetic average roughness Ra to DIN 4768 of press mold surfaces should preferably be 0.01 to 0.5 μm, more preferably 0.02 to 0.3 μm, even more preferably 0.02 to 0.2 μm, most preferably 0.02 to 0.1 μm.

The length of the tip, specifically of the lower punch, is preferably 2 to 7 mm, more preferably 2 to 6 mm, most preferably 2.5 to 5 mm. Excessively high tip lengths can lead to high friction, especially when sticking of the shaped precursor body occurs. It is preferable that the upper and lower edges of the tip, specifically of the lower jaw, are not rounded but sharp. The sharp edge alleviates clogging of the powder in the margins at the stamp-stamp interface and the pin-hole interface (in the case of tablet molds with passages). Powder clogging leads both to sticking and to escape of powder.

The length of the tip specifically of the upper punch is preferably greater than 2 mm and is typically in the range from 2 to 10 mm. By contrast with the lower punch, a high tip length does not cause friction problems since the upper punch is introduced only a few millimeters into the die in the tableting cycles.

If the tableting mold has passages, the lower punch and the upper punch have holes to accommodate the pins. The upper punches should have at least one ventilation hole that enables the air to escape from the die cavity through the upper stamp holes to the outside of the upper stamp holes during the compaction. Such upper punches with ventilation opening hole(s) are specified in US 2010/0010238 (see FIGS. 4a, 4b, 4c and 4d).

The distance between the nozzle bore and the lower outer face of the stamp is preferably 3 to 50 μm, more preferably 5 to 35 μm, most preferably 6 to 26 μm. Analogously, the distance between the die bore and the upper outer face of the stamp is preferably 3 to 50 μm, more preferably 5 to 35 μm, most preferably 6 to 26 μm. The distance is ensured by the selection of a suitable combination of dimensional tolerances of the die and the lower punch. The dimensional tolerance is typically illustrated by the ISO wave tolerance defined in ISO 286-2. Examples of combinations of dimensional tolerance of the nozzle bore and the outside of the punch that are illustrated in ISO tolerance codes are H6/f7, H6/g6, H6/g7, H7/g6, H7/f7, F8/h6, G7/h6, F7/h6 (nozzle bore/lower outer face of punch).

In cases where the tablet shape has passages, the press mold comprises pins. The distance between the pin bore of the lower punch and the pin is preferably 3 to 50 μm, more preferably 5 to 35 μm, most preferably 6 to 26 μm. Analogously to the distance between the die bore and the outside of the stamp, this distance is ensured via selection of a suitable combination of dimensional tolerances of the die and lower punch.

The die bore preferably has a slight constriction that starts from a defined depth in the direction of the top end of the die. The narrowing portion of the nozzle bore has a gradual increase in bore size in the direction of the top end of the nozzle, which gives rise to an additional distance between the nozzle bore wall and the tipped outer lateral face of the lower punch. The additional space facilitates escape of the air present in the mixing feed during the compression in the die, which alleviates blowing of the powder and resultant unstable tableting owing to poor escape of air. A further advantage of conical dies is facilitation of ejection after compaction. Compaction to a tablet at the point where the die bore has a constriction results in a tablet having a slightly constricted outer lateral face owing to the shaping by the conical die bore. In the ejection phase, in which the lower punch pushes the tablet upward in the die, the tablet is easily ejected from the die wall since slight raising of the tablet forces detachment of the tablet from the die wall owing to the narrowing structure. When the die bore does not have a constriction, detachment of the tablet from the die wall does not occur, and so the entire ejection process (i.e. the raising of the tablet from the depth at which compression on the top side of the nozzle has occurred) suffers from friction between the outer surface of the tablet and the die wall and between the straight outer face of the tip and the nozzle wall. The friction leads to an unfavorably high ejection force.

The depth (i.e. depth from the top side of the die) of the matrix constriction should be chosen such that the tablet formed, prior to ejection, is predominantly in the conical zone. In order to achieve such a situation, the depth of the die constriction may be oriented by adding up the height of the in-die tablet prior to elastic recovery (i.e. minimum distance between the upper and lower punches) and the insertion depth of the upper punch. For example, a constriction depth of 12 to 15 mm may be used for an in-die tablet height of 12 mm with an insertion depth of 2 mm of the upper punch.

The angle of the die constriction is typically 0.1 to 0.6°, and the increase in size of the die bore on the upper side is preferably 0.03 to 0.2 mm, more preferably 0.05 to 0.14 mm. The increase in size can be derived mathematically from the angle of constriction and the constriction depth.

In cases where the tablet shape has passages, the press mold comprises pins. The pins are secured on the revolver, such that the pins are present in the die cavity in which the tablet is formed, in order to leave the passages of the tablet. Analogously to the die, the pins do not move vertically during the tableting cycle, by contrast with the upper and lower punches. The vertical plane of the upper end of the pins is the same as or slightly below the height of the upper end face of the die. Especially in cases where the tablet has curved lateral faces, in which the lower stamp face has a concave surface, the vertical plane of the upper end of the pin should be somewhat below the level of the upper front side of the die, in order that the pins do not project from the lower stamp face.

In cases in which the tablet shape has passages, it is frequently sticking on the surface of the pins, which leads to drawbacks, for example high ejection force owing to high friction at pin-tablet interfaces. The problem is particularly marked in the case of multiple molds with several pins. Typically, the pins have a higher tendency to sticking than the die wall and the tip specifically of the lower jaw.

In cases where the tablet shape has passages, the pins preferably have a slight constriction in the upper region for a defined length. The narrowing portion of the pins shows a gradual decrease in pin diameter toward the upper end of the pin. The main advantage of the conical pins is facilitation of ejection after compaction. Compaction to a tablet at the place where the pins have a constriction leads to tablet passages having a slightly constricted inner lateral face as a result of the shaping by the narrowing pins. The diameters of the tablet passages decrease slightly from the bottom upward along the axial axis. In the ejection phase, in which the lower punch pushes the tablet upward in the die, while the pins and the die remain vertically immobile, the tablet is ejected easily from the pins, since slight raising of the tablet forces detachment of the tablet from the pins owing to the narrowing structure. If the pins have no constriction, detachment of the tablet from the pins does not occur, and so the entire ejection process (i.e. the lifting of the tablet from the depth at which compression was effected on the upper end face of the die) suffers from friction at the tablet-pin interfaces, which leads to a high ejection force. The conical pins are particularly advantageous when the tablet has multiple passages.

The length of the constriction of the pins should be chosen such that the tablet formed, prior to ejection, is predominantly in the conical zone. In order to achieve such a situation, the length of the constriction of the pins may be oriented by adding up the height of the in-die tablet prior to elastic recovery (i.e. minimum distance between the upper and lower punches) and the introduction depth of the upper punch. For example, a narrowing length of 12 to 15 mm may be used for an in-die tablet height of 12 mm with an insertion depth of 2 mm of the upper punch.

The angle of the die constriction is typically 0.1 to 0.6°, and the reduction in pin diameter on the upper side is preferably 0.05 to 0.3 mm, more preferably 0.1 to 0.2 mm. The reduction can be derived mathematically from the angle of constriction and the constriction length.

Industrial mass production of tablets is preferably effected on a rotary tableting press. Conventional rotary presses may be used for this invention. Examples of rotary tableting presses are Korsch XT-600 HD, Korsch XT-600, Korsch TPR 700, Korsch TRP 1200, Korsch XL 400 MFP, Kilian RX and Kilian Synthesis.

Rotary presses typically have two compaction rolls in order to conduct a two-stage compaction, comprising a preliminary compaction and a main compaction. The main compaction pressure is in the range from 5 to 500 MPa, preferably 8 to 400 MPa, more preferably 10 to 300 MPa. The preliminary compaction pressure is typically in the range from 5% to 50%, preferably 7% to 40%, more preferably 10% to 35%, of the main compaction pressure applied.

The press mold is selected in accordance with the desired geometric dimensions of the compacted body. The size and shape of the compacted body and hence of the catalyst is chosen such that suitable packing of the catalyst bodies obtained from compacted bodies in a reactor tube is possible. The catalysts obtained from the compacted bodies suitable for the catalysts of the invention are preferably used in reactor tubes having a length of 6 to 14 m and an internal diameter of 20 mm to 50 mm. In general, the support consists of individual bodies having a maximum extent in the range from 3 to 20 mm, such as 4 to 15 mm, especially 5 to 12 mm. The maximum extent means the longest straight line between two points on the outer circumference of the support.

The shape of the compacted bodies is not particularly limited and may be any technically conceivable shape, depending on the shaping process for example. For example, the assistance may be a solid tablet or a hollow tablet, such as a hollow cylinder. In a further embodiment, the pillar may be characterized by a multilobal structure. A multilobal structure is supposed to refer to a cylinder structure having a multitude of cavities, e.g. grooves or flutes, that run in the periphery of the cylinder along the cylinder height. In general, the cavities are in an essentially equidistant arrangement around the circumference of the cylinder.

The compression force on tableting influences the compaction of the free-flowing feed mixture and hence, for example, the density and/or mechanical stability of the compressed body. In practice, it has been found to be advisable to control the lateral compressive strength of the tableted catalyst support by selection of the suitable compression force and to verify it by sampling. For the purposes of the present invention, the lateral compression force is the force that breaks the tableted catalyst pillar between two flat parallel plates, where the two flat parallel end faces of the catalyst pillar are at right angles to the flat parallel plates.

In order to improve tableting properties, the free-flowing feed mixture may be subjected to further processing, for example by sieving, preheating and/or pre-pelletization, i.e. precompaction. For the pre-pelletization, it is possible to use a roll compressor, for example a Chilsonator® from Fitzpatrick.

Further information on tableting, especially on pre-pelletization, sieving, lubricants and molds, can be found in WO 2010/000720. You can find further information on tableting in the Handbook of Powder Technology, Chapter 16: Tabletting, K. Pitt and C. Sinka, Vol 11, 2007, p. 735 to 778.

Advantageously, a tableting operation is conducted as described in documents WO 2005/030393, DE 10 2008 040093 A1, DE 10 2008 040094 A1 and WO 2007/017431. The ambient temperature around the tableting machine is normally 25 °C. The particle diameters of the precursor composition to be compacted (of the finely divided intimate dry mixture), optionally as a result of prior coarsening by compaction, are appropriately in the range of 100 to 2000 μm, preferably 150 to 1500 μm, more preferably 400 to 1250 μm, or 400 to 1000 μm, or 400 to 800 μm (shaping auxiliary mixed in prior to the compaction is not taken into account here).

Just like the shaping apparatus to be used for compaction or the shaping method to be employed here, the desired geometry of the resulting shaped bodies in the process of the invention is not subject to any restriction either.

A preferred ring geometry (the geometry of an uncalcined green body and of the shaped unsupported catalyst body that arises therefrom through calcination are normally essentially the same) is the geometry of 5 mm×5 mm×2 mm (external diameter×height (length)×internal diameter). This is because fixed catalyst beds composed of rings of this geometry result in a particularly small pressure drop for the reaction gas mixture flowing through the fixed catalyst bed (especially in reaction tubes with an internal diameter of 22 mm to 30 mm). A small pressure drop is advantageous especially when the partial oxidation catalyzed by the fixed catalyst bed is being operated at a high space velocity of reaction gas mixture on the fixed catalyst bed (at a high flow rate of the reaction gas mixture flowing through the fixed catalyst bed). Another preferred ring geometry (which especially has advantageous bulk characteristics in reaction tubes with relatively small internal diameter (for example 20 mm)) is the geometry 5 mm×3 mm×2 mm (external diameter×height (length)×internal diameter). Useful geometries are of course alternatively those that are disclosed and recommended in WO 02/062737 and WO 2015/067656.

Especially in the case of annular shaped precursor bodies (irrespective of their shape, shaped precursor bodies are also referred to in the literature as green bodies), the shaping compaction should advantageously be conducted such that the side crushing strength SDFᵥ of the resulting shaped body (cf. DE 10 2008 040093 A1, DE 10 2008 040094 A1 and WO 2005/030393) satisfies the relation 5 N≤SDFᵥ≤100 N, preferably 8 N≤SDFᵥ≤80 N, and more preferably 12 N≤SDFᵥ≤50 N.

The experimental determination of side crushing strength is conducted as described in documents WO 2005/030393 and WO 2007/017431. It will be appreciated that ringlike green bodies as recommended by DE 10 2008 040093 A1 are very particularly preferred in accordance with the invention. The end faces of annular or ringlike shaped bodies, in the described production process for green bodies of the invention, may be either planar (both or just one of the two end faces) or domed (curved) outward (convexly) (cf., in particular, DE 10 2007 004961 A1, EP 0 184 790 A2, DE 10 2008 040093 A1 (for example paragraph [0032] thereof) and DE 10 2008 04009 A1 (for example paragraph [0074] thereof) and the embodiments described in individualized form in these documents). In the determination/specification of the height of such geometric shaped bodies, such a convex dome (curvature) is generally not taken into account in the present document. Rings or ringlike shaped bodies with convex (curved) end faces (preferably both end faces have the same curvature) are advantageous in that fixed catalyst beds of annular or ringlike shaped bodies with convex (curved) end face (with otherwise identical geometry) result in a lower pressure drop in the reaction gas mixture flowing through the fixed catalyst bed (in particular in the case of fixed catalyst beds in reaction tubes) than fixed catalyst beds of annular or ringlike shaped bodies with a planar end face. This is true especially when the ring geometry is 5 mm×5 mm×2 mm (external diameter×height (length)×internal diameter). The radius of such a convex curvature is generally 0.4 to 5 times (e.g. 0.8 to 4 times, or 1.2 to 3 times, or 1.6 to 2.6 times) the external diameter of the circular cylinder of the catalyst ring. As already mentioned, a fixed catalyst bed that results in a smaller pressure drop is advantageous especially when the partial oxidation catalyzed by the fixed catalyst bed is being operated at a high space velocity of reaction gas mixture on the fixed catalyst bed (at a high flow rate of the reaction gas mixture flowing through the fixed catalyst bed).

Particularly advantageous ring geometries of shaped bodies obtainable by compacting finely divided precursor composition (finely divided intimate dry mixture) satisfy the condition Height (length)/External diameter=H/E=0.3 to 1.5 or to 1.2. More preferably, H/E=0.5 to 1.1 or to 1.0. In addition, it is favorable for annular or ringlike green bodies of the invention when the ratio I/E (where I is the Internal diameter of the ring geometry) is 0.3 to 1.5, preferably 0.6 to 1.1.

The aforementioned ring geometries are particularly advantageous when they simultaneously have one of the advantageous H/E ratios and one of the advantageous I/E ratios. Such possible combinations are, for example, H/E=0.3 to 1.5 or to 1.2 and I/E=0.3 to 1.5 or 0.6 to 1.1. Alternatively, H/E may be 0.5 to 1.1 or to 1.0, and I/E may simultaneously be 0.3 to 1.5 or 0.6 to 1.1. In addition, it is favorable for the relevant ring geometries when H is 2 to 7 mm and preferably 2 to 6 mm or 3 to 6 mm. It is also advantageous when E in the rings is 4 to 8 mm, preferably 4 to 6 mm. The wall thickness of preferred ring geometries is 1 to 2 mm or to 1.5 mm.

Possible ring geometries of the invention are thus (E×H×1) 5 mm×5 mm×2 mm, or 5 mm×2 mm×2 mm, or 5 mm×3 mm×2 mm, 5 mm×5 mm×2.5 mm, or 5 mm×3 mm×2.5 mm, or 5.5 mm×5.5 mm×2.5 mm, or 5.5 mm×5.5 mm×3 mm, or 5 mm×3 mm×3 mm, or 5.5 mm×3 mm×3.5 mm, or 6 mm×3 mm×4 mm, or 6 mm×6 mm×3 mm, or 6 mm×6 mm×3.5 mm, or 6.5 mm×3 mm×4.5 mm, or 7 mm×3 mm×5 mm, or 7 mm×7 mm×3 mm, or 7 mm×3 mm×4 mm, or 7 mm×7 mm×4 mm.

All FIGURES in this document for specific surface areas of solids relate to determinations to DIN 66131 (determination of the specific surface area of solids by gas adsorption (N₂) according to Brunauer-Emmett-Teller (BET)), unless explicitly stated otherwise.

All FIGURES in this document for total pore volumes and for pore diameter distributions over these total pore volumes relate to determinations by the method of mercury porosimetry employing the Auto Pore V 9600 system (MicroActive Interactive Dara Analysis Software) from Micromeritics GmbH, D-52072 Aachen (range: 0.1-61 000 psi) at 23 °C (evaluation by Washburn equation using a contact angle of mercury of 140° and a surface tension of mercury of 480 mN/m=480 dyn/cm).

Advantageously in accordance with the invention, shaped precursor bodies have a minimum residual moisture content. This is especially true when the intimate mixing of the various sources of the elemental constituents other than oxygen was effected in wet form (especially when an aqueous mixture M was formed).

The residual moisture content of advantageous green bodies preferably has values of ≤10% by weight, better ≤8% by weight, even better ≤6% by weight, and at best ≤4% by weight or ≤2% by weight (residual moisture content can be determined as described in “Die Bibliothek der Technik” [The Library of Technology], volume 229, “Thermogravimetrische Materialfeuchtebestimmung”, Grundlagen und praktische Anwendungen [“Thermogravimetric Determination of Material Moisture Content”, Basics and Practical Applications], Horst Nagel, verlag moderne industrie (for example with the aid of a Computrac MAX 5000 XL from Arizona Instruments)).

If the green bodies originate from an aqueous mixture M (such that the residual moisture content thereof consists of water), the residual moisture content is determined, appropriately for application purposes, with microwaves (e.g. with the LB 456 microwave system from BERTHOLD TECHNOLOGIES).

In this procedure, the microwave irradiates the material to be examined with very low power (0.1 mW) (the material undergoes essentially no change in temperature as a result of the comparatively low power). The material constituents are polarized to different degrees as a result. In reaction, the microwave loses speed and energy. The influence of water molecules here is significantly greater than the influence of other constituents, which enables the selective determination of residual water contents. This is because water molecules, on account of their size and their dipole capacity, have particularly good ability to follow an electromagnetic alternating field in the microwave frequency region by dipole alignment. In so doing, they absorb energy and alter the electromagnetic alternating field with their electrical properties. This field attenuation and field alteration is the basis of the measurement principle. For example, it is possible to build up a weak microwave field across the sensor area of a planar sensor and permanently analyze the resonance frequency of the sensor system by scanning the microwave frequency. If a water-containing measurement material is moved across the sensor, there is a shift in the resonance frequency and attenuation of the amplitudes thereof. There is a rise both in damping and shift in resonance frequency with increasing amount of water, i.e. with increasing bulk density of the measurement material. However, the ratio of shift in frequency and damping is a density-independent measure of the percentage water content and hence the key to the measurement of moisture content. The ratio forms what is called the microwave moisture measurement, which represents the total moisture content. Since the microwave resonance method is an indirect moisture measurement method, calibration is necessary. In such a calibration measurement, the sensor is used to measure material samples with defined moisture content. The relationship of the microwave moisture measurements to the corresponding defined absolute material moisture contents then forms the calibration of the measurement system. Measurement accuracy is typically ±0.1% moisture content (for example, the water moisture content can be determined with a Sartorius PMD300PA online moisture analyzer).

Against this background, spray drying of a wet (e.g. aqueous) mixture M should already be conducted such that the resulting spray powder has a minimum residual moisture content.

The green bodies, taking account of the aspect just addressed, should be stored as far as possible with exclusion of ambient air (having air humidity) (preferably, storage until calcination is effected under anhydrous inert gas or under predried air or in airtight containers).

The forming/shaping and storage of finely divided intimate dry mixture is advantageously conducted with exclusion of ambient air (having air humidity) (for example under N₂ atmosphere).

The green bodies are normally calcined at temperatures (calcination temperatures) that attain or generally exceed at least 350 °C. Normally, however, the temperature of 650 °C is not exceeded in the course of calcination (the term “calcination temperature” in this document means the temperature present in the calcination material (the calcination material advantageously has a very uniform (homogeneous) calcination temperature; this is correspondingly true of the other calcination conditions)). Advantageously, in the course of calcination, the temperature of 600 °C, preferably the temperature of 570 °C and frequently the temperature of 550 °C is not exceeded. Moreover, in the course of the above calcination, preferably the temperature of 380 °C, advantageously the temperature of 400 °C, particularly advantageously the temperature of 420 °C and most preferably the temperature of 440 °C is exceeded. The calcination may also be subdivided into multiple sections over its duration.

Advantageously, the calcination is preceded by a thermal pretreatment at temperatures of ≥120 °C and <350 °C, preferably ≥150 °C and ≤320 °C, more preferably ≥170 °C and ≤290 °C. Appropriately for application purposes, such a thermal pretreatment is conducted until the constituents that are present within the composition to be subjected to thermal treatment and break down to gaseous compounds under the conditions of the thermal treatment have largely (preferably completely) broken down to give gaseous compounds (the time taken in this regard may, for example, be 3 to 15 hours, frequently 4 to 10 hours or 5 to 8 hours). This is generally the case when, firstly, the molar amount of cations other than metal ions present in the composition which is subsequently to be calcined, based on the total molar amount of cations present, is ≤20 mol % (preferably ≤10 mol %) and, secondly, the molar amount of anions other than O₂⁻ present in the same composition, based on the total molar amount of anions present, is likewise ≤20 mol % (preferably ≤10 mol %).

Favorable temperature windows for the final calcination temperature are therefore within the temperature range of 400 to 600 °C, or preferably in the temperature range of 420 to 570 °C, or more preferably in the temperature range of 450 to 550 °C.

The total calcination time generally extends to more than 0.5 hour, and frequently to more than 2 hours. Usually, in the calcination, treatment times of 45 hours, or of 30 hours, are not exceeded. The total calcination time is often below 25 hours. In principle, a shorter calcination time is generally sufficient at higher calcination temperatures than at lower calcination temperatures. In one embodiment of the calcination which is advantageous in accordance with the invention, 550 °C is not exceeded, and the calcination time in the temperature window of ≥430 °C and ≤550 °C extends to >4 to ≤25 hours.

The overall thermal treatment (including a breakdown phase) of a precursor composition (for example a green body) can be effected either under inert gas or under an oxidative atmosphere, for example air (or another mixture of inert gas and molecular oxygen) and under a reducing atmosphere (for example a mixture of inert gas, NH₃, CO and/or H₂, or under methane). The thermal treatment can of course also be executed under reduced pressure. The atmosphere can also be varied over the course of the thermal treatment.

The thermal treatment (especially the calcination phase) is preferably effected in an oxidizing atmosphere. Appropriately in application terms, this atmosphere consists predominantly of stationary air or (preferably) of moving air (more preferably, an air stream flows through the composition to be subjected to thermal treatment (the calcination material)). However, the oxidizing atmosphere may likewise consist of a stationary or moving mixture of, for example, 25% by volume of N₂ and 75% by volume of air, or 50% by volume of N₂ and 50% by volume of air, or 75% by volume of N₂ and 25% by volume of air (a treatment atmosphere composed of 100% by volume of N₂ is likewise possible).

In principle, the thermal treatment (for example the calcination) of the precursor composition (for example of the green bodies) can be conducted in a wide variety of different oven types, for example heatable air circulation chambers (air circulation ovens, for example air circulation shaft ovens), staged ovens, rotary ovens, belt calciners or shaft ovens. Advantageously in accordance with the invention, the thermal treatment (for example the calcination) is effected in a belt calciner apparatus as recommended by DE 100 46 957 A1 and WO 02/24620. Hotspot formation within the material to be treated (within the calcination material) is very substantially prevented here in that, with the aid of ventilators, elevated volume flow rates of calcination atmosphere are conveyed through the calcination material on a gas-permeable conveyor belt that bears the calcination material (these assure very substantial homogeneity of the calcination temperature in the calcination material).

In the thermal treatment of the precursor compositions to be conducted as described (for example of the green bodies), shaping auxiliaries used can either be preserved in the resulting shaped catalyst body or at least partly escape in gaseous form therefrom as a result of thermal and/or chemical breakdown to give gaseous compounds (e.g. CO, CO₂). Shaping auxiliaries remaining in the shaped catalyst body, in the course of a catalytic use thereof, have essentially an exclusively diluting effect on the active composition. In principle, the thermal treatment in this regard can be effected as described in US 2005/0131253.

Typically, the side crushing strengths of annular shaped unsupported catalyst bodies obtainable as described in accordance with the invention are 4 to 16 N, frequently 5 to 14 N or 6 to 12 N.

The specific (BET) surface area of shaped unsupported catalyst bodies is advantageously 2 to 20 m²/g or to 15 m²/g, preferably 3 to 10 m²/g and more preferably 4 to 8 m²/g. Advantageously in accordance with the invention, the corresponding total pore volume (mercury porosimetry) is in the range from 0.33 to 0.60 cm³/g, preferably in the range of 0.36 to 0.54 cm³/g and more preferably in the range of 0.38 to 0.50 cm³/g.

If the pore diameter in μm is plotted on the abscissa and the logarithm of the differential contribution in cm³/g of the respective pore diameter to the total pore volume in cm³/g on the ordinate, shaped unsupported catalyst bodies that are particularly favorable in accordance with the invention generally show an essentially monomodal distribution (have only one pronounced maximum). If the contribution of pores having a pore radius of ≤0.1 μm to the total pore volume is ≤0.05 cm³/g, the result is particularly good overall target product selectivities (for example in the case of a heterogeneously catalyzed partial oxidation of propene to acrolein and/or acrylic acid). If the contribution of such comparatively narrow pores to the total pore volume is >0.05 cm³/g, an increase in the calcination time and/or the calcination temperature can bring about a reduction in this contribution which is advantageous in accordance with the invention.

Furthermore, for an elevated overall target product selectivity, it is found to be advantageous when the contribution of pores having a pore radius in the range of 0.1 to 1 μm to the total pore volume, based on the total pore volume, is 85% to 99% by volume, advantageously 87% to 97% by volume, more preferably 89% to 95% by volume.

It will be appreciated that the shaped unsupported catalyst bodies may also be used diluted with inert materials for catalysis of heterogeneously catalyzed partial gas phase oxidations. Suitable inert diluent materials of this kind include element oxides that have been calcined at high temperatures and hence have a comparatively low level of pores, such as aluminum oxide, silicon dioxide, thorium dioxide and zirconium dioxide. Alternatively, finely divided silicon carbide or finely divided silicates such as magnesium silicate and aluminum silicate or steatite may be used for the aforementioned purpose. Advantageously for application purposes, the procedure here will be, for example, to grind the calcined active composition to a finely divided powder. This is then mixed with finely divided diluent material, and the resulting mixed powder is formed using a shaping method presented in this document (preferably by tableting) to a geometric shaped body. By subsequently calcining once again, the latter is then transformed to the corresponding shaped unsupported catalyst body. The finely divided inert diluent material can of course, however, already be incorporated, for example, into a wet (for example aqueous) mixture M prior to the drying thereof. In addition, finely divided inert diluent material can be incorporated into the finely divided dry mixture. However, such procedures are less preferred in accordance with the invention.

It is a particular feature of shaped unsupported catalyst bodies produced by the advantageous production methods described that they have essentially no local centers of element oxides. Instead, these elements are very substantially part of complex mixed oxomolybdates comprising Bi, Fe and Mo. This is found to be favorable with regard to the inventive aim of minimization of full combustion of organic reaction gas mixture constituents, which is undesirable in accordance with the invention, in the course of the relevant heterogeneously catalyzed partial oxidations.

Otherwise, the procedure for production of unsupported catalysts, from the point of view of maximum efficiency of material utilization, is appropriately as in WO 2010/066645.

Inventive shaped unsupported catalyst bodies are suitable not just for catalysis of the heterogeneously catalyzed partial oxidation of propene to acrolein but generally for catalysis of heterogeneously catalyzed partial gas phase oxidations of alkanes, alkanols, alkenes and/or alkenals having 3 to 6 carbon atoms (partial oxidations shall be understood in this document especially to mean those conversions of organic compounds with reactive involvement of molecular oxygen in which the organic compound to be partially oxidized, after the reaction has ended, comprises at least one oxygen atom more in chemically bound form than prior to performance of the partial oxidation). The term “partial oxidation” in this document shall alternatively comprise oxidative dehydrogenation and partial ammoxidation, i.e. a partial oxidation in the presence of ammonia.

Shaped unsupported catalyst bodies of the invention are particularly suitable for catalysis of the heterogeneously catalyzed partial gas phase oxidation of propene to acrolein, of isobutene to methacrolein, and for catalysis of the heterogeneously catalyzed partial gas phase ammoxidation of propene to acrylonitrile and of isobutene to methacrylonitrile.

As already mentioned, the heterogeneously catalyzed partial gas phase oxidation of propene (isobutene and/or tert-butanol) to acrolein (methacrolein) forms the first stage of a two-stage heterogeneously catalyzed partial gas phase oxidation of propene (isobutene and/or tert-butanol) to acrylic acid (methacrylic acid), as described by way of example in WO 2006/42459.

Formation of acrylic acid (methacrylic acid) by-product associated with a heterogeneously catalyzed partial gas phase oxidation of propene (isobutene) to acrolein (methacrolein) is therefore generally not undesirable and is normally covered by the desired formation of products of value.

The above is especially true of annular shaped unsupported catalyst bodies of the invention.

The heterogeneously catalyzed partial oxidation (especially that of propene to acrolein) can be conducted as described, for example, in documents DE 10 2007 004961 A1, WO 02/49757, WO 02/24620, DE 10 2008 040093 A1, WO 2005/030393, EP 0 575 897 A1, WO 2007/082827, WO 2005/113127, WO 2005/047224, WO 2005/042459, WO 2007/017431, DE 10 2008 042060 A1, WO 2008/087116, DE 10 2010 048405 A1, DE 10 2009 047291 A1, DE 10 2008 042064 A1, DE 10 2008 042061 A1, WO 2015/067656 and DE 10 2008 040094 A1 for similar catalysts (more particularly, it is possible here to proceed in a corresponding manner to that in the illustrative embodiments of these documents).

However, shaped unsupported catalyst bodies (especially the annular shaped unsupported catalyst bodies) that are obtainable as described are also advantageous when the space velocity of propene, isobutene and/or tert-butanol (or the methyl ether thereof) present in the reaction gas input mixture on the catalyst charge of a reactor is ≥130 L (STP)/L of catalyst charge·h, or ≥140 L (STP)/L·h, or ≥150 L (STP)/L·h, or ≥160 L (STP)/L·h (upstream and/or downstream beds of pure inert material are not considered to form part of the catalyst charge in considerations of space velocity in this document; the volume of the catalyst charge (of the fixed catalyst bed) is incidentally the bed volume thereof within the reactor). Normally, the aforementioned space velocity of the catalyst charge will be ≤600 L (STP)/L·h, frequently ≤500 L (STP)/L·h, in many cases ≤400 L (STP)/L·h or ≤350 L (STP)/L·h. Space velocities in the region of ≥160 L (STP)/L·h or ≥180 L (STP)/L·h to ≤300 or ≤250 or ≤200 L (STP)/L·h are particularly appropriate.

In this document, the space velocity of reaction gas input mixture on a fixed catalyst bed is understood to mean the amount of reaction gas input mixture in standard liters (=L (STP); the volume in liters that the corresponding amount of reaction gas input mixture would occupy under the standard conditions of 0 °C and 1 atm (1.01 bar)) which is supplied to the fixed catalyst bed, based on the volume of its bed (not including bed sections of pure inert material), i.e. on its bed volume, per hour (−>unit=L (STP)/L·h). In this document, the units of standard volume such as L (STP) or m³ (STP) always relate (unless explicitly stated otherwise) to the standard conditions of 0 °C and 1 atm (1.01 bar).

The space velocity may also be based only on one constituent of the reaction gas input mixture (for example only on the organic starting compound to be partially oxidized). In that case, it is the volume of this constituent (for example of the organic starting compound of the partial oxidation) which is supplied to the fixed catalyst bed (the catalyst charge of the reactor), based on the volume of its bed, per hour.

Shaped unsupported catalyst bodies (for example in annular form) that are obtainable in accordance with the invention may of course also be operated in a manner advantageous in accordance with the invention as catalysts for the partial oxidation of propene to acrolein or of isobutene and/or tert-butanol (or the methyl ether thereof) to methacrolein at space velocities of the starting compound to be partially oxidized on the catalyst charge of ≤130 L (STP)/L·h, or ≤120 L (STP)/L·h, or ≤110 L (STP)/L·h, or ≤100 L (STP)/L·h, or ≤90 L (STP)/L·h. In general, however, this space velocity will be at values of ≥20 L (STP)/L·h, or ≥30 L (STP)/L·h, or ≥40 L (STP)/L·h, or ≥50 L (STP)/L·h, or ≥60 L (STP)/L·h, or ≥70 L (STP)/L·h, or ≥80 L (STP)/L·h.

In principle, the space velocity of the starting compound to be partially oxidized (propene, isobutene and/or tert-butanol (or the methyl ether thereof)) on the catalyst charge of a reactor (on a fixed catalyst bed) can be adjusted via two adjusting screws:
a) the space velocity of reaction gas input mixture (the reaction gas mixture which is supplied to the fixed catalyst bed) on the catalyst charge, and/or
b) the content of the starting compound to be partially oxidized in the reaction gas input mixture.

The shaped unsupported catalyst bodies (for example in annular form) that are obtainable in accordance with the invention are also suitable especially when, at space velocities of the organic compound to be partially oxidized on the catalyst charge above 130 L (STP)/L·h, the space velocity is adjusted via the aforementioned adjustment screw a) in particular.

In general, for example, the propene content (isobutene content or tert-butanol content (or the methyl ether content)) in the reaction gas input mixture, essentially irrespective of the space velocity on the catalyst charge, will be 4% to 10% by volume, frequently 5% to 9% by volume, or 5.5% to 8.0% by volume, or 6.0% to 7.5% by volume (based in each case on the total volume (flow rate) of the reaction gas input mixture flowing toward the fixed catalyst bed).

Frequently, the gas phase partial oxidation process of the partial oxidation catalyzed by the shaped unsupported catalyst bodies of the invention (for example in annular form) that are obtainable as described will be conducted (essentially irrespective of the space velocity) at a volume ratio of (organic) compound to be partially oxidized (for example propene):oxygen:inert gases (including steam) in the reaction gas input mixture of 1:(1.0 to 3.0):(5 to 25), preferably 1:(1.5 to 2.3):(10 to 20).

Inert gases are understood here to mean those gases which, in the course of the partial oxidation, in a single pass of the reaction gas mixture through the catalyst bed, remain chemically unchanged to an extent of at least 95 mol %, preferably to an extent of at least 98 mol %.

In the above-described reaction gas input mixtures, the inert gas may consist of molecular nitrogen to an extent of ≥20% by volume, or to an extent of ≥30% by volume, or to an extent of ≥40% by volume, or to an extent of
≥50% by volume, or to an extent of ≥60% by volume, or to an extent of ≥70% by volume, or to an extent of ≥80% by volume, or to an extent of
≥90% by volume, or to an extent of ≥95% by volume.

However, at higher space velocities of the organic compound to be partially oxidized on the catalyst charge of the reactor (for example of ≥150 L (STP)/L·h), the additional use of inert diluent gases having elevated molar heats and/or thermal conductivities, such as propane, ethane, methane, pentane, butane, CO₂, CO, steam and/or noble gases is advisable (but not obligatory) for the reaction gas input mixture. In general, these inert gases and mixtures thereof may also be used even at lower space velocities of the organic compound to be partially oxidized on the catalyst charge. Cycle gas can also be used in part as diluent gas. Cycle gas is understood to mean the tail gas that remains when the target compound is separated essentially selectively from the product mixture of the partial oxidation. It should be taken into account here that the partial oxidations to acrolein or methacrolein with the shaped unsupported catalyst bodies, for example in annular form, that are obtainable in accordance with the invention may be just the first stage of a two-stage partial oxidation to acrylic acid or methacrylic acid as the actual target compounds, such that the cycle gas is usually formed only after the second stage (typical cycle gas compositions in the case of a heterogeneously catalyzed partial oxidation of propene to acrolein and/or acrylic acid are shown in DE 102 32 482 A1 in paragraphs [0063] and [0075] thereof). In the context of such a two-stage partial oxidation, in general, the product gas mixture from the first stage is fed as such, optionally after cooling and/or secondary oxygen addition (generally in the form of air), to the second partial oxidation stage.

In the partial oxidation of propene to acrolein, using the shaped unsupported catalyst bodies of the invention (for example in annular form) that are obtainable as described, a typical composition of the reaction gas input mixture measured at the reactor inlet (irrespective of the space velocity chosen) may comprise the following components for example:
6% to 6.5 vol % of propene,
1% to 3.5 vol % of H₂O,
0.8% to 1.7 vol % of COₓ,
0.015% to 0.04 vol % of acrolein,
9.4% to 12.3 vol % of oxygen, and
as the remainder to 100 vol %, molecular nitrogen;
   or
5.6 vol % of propene,
1.4 vol % of H₂O,
1.2 vol % of COₓ,
10.2 vol % of oxygen, and
as the remainder to 100 vol %, molecular nitrogen;

The former compositions are especially suitable at propene space velocities of ≥130 L (STP)/L·h, and the latter composition especially at propene space velocities of <130 L (STP)/L·h, especially ≤100 L (STP)/L·h (for example for startup of the partial oxidation), on the fixed catalyst bed.

Alternatively, it is also possible to use reaction gas input mixtures of the composition according to Example 1 of EP 0 990 636 A1, or according to example 2 of EP 0 990 636 A1, or according to example 3 of EP 1 106 598 A2, or according to example 26 of EP 1 106 598 A2, or according to example 53 of EP 1 106 598 A2, or according to the examples of WO 2021/013640 for an inventive propene partial oxidation to acrolein.

The shaped unsupported catalyst bodies of the invention, for example in annular form, that are obtainable as described are also suitable for the processes of DE 102 46 119 A1 or DE 102 45 585 A1.

The reaction temperature for an inventive heterogeneously catalyzed propene partial oxidation to acrolein, in the case of use of the shaped unsupported catalyst bodies of the invention (for example in annular form) that are obtainable as described, is frequently 300 to 450 °C, or to 400 °C, or to 380 °C. A particularly preferred reaction temperature window is 305 to 345 °C. The same is true in the case of methacrolein as target compound.

The reaction pressure for the aforementioned partial oxidations is generally 0.5 to 4 or to 3 bar, or preferably 1.1 or 1.5 to 4 or to 3 bar (what are meant in this document, unless explicitly stated otherwise, are always absolute pressures).

The total space velocity of reaction gas input mixture on the catalyst charge in the aforementioned partial oxidations of the invention typically runs to 1000 to 10 000 L (STP)/L·h, usually to 1500 to 5000 L (STP)/L·h and often to 2000 to 4000 L (STP)/L·h.

Useful propene for use in the reaction gas input mixture is, in particular, polymer grade propene and chemical grade propene as described, for example, by WO 2004/007405.

The oxygen source used is normally air (optionally together with cycle gas).

The partial oxidation using the shaped unsupported catalyst bodies (for example the annular shaped unsupported catalyst bodies) of the invention that are obtainable as described can be conducted in the simplest case, for example, in a single-zone multiple catalyst tube fixed bed reactor as described in DE 44 31 957 A1, EP 0 700 714 A1 and EP 0 700 893 A1.

Customarily, in the aforementioned shell and tube reactors, the catalyst tubes are manufactured from ferritic steel and typically have a wall thickness of 1 to 3 mm. Their internal diameter is generally from 20 to 30 mm, frequently from 21 to 26 mm. A typical catalyst tube length is, for example, 3.50 m, 4.00 m or 4.50 m. The number of catalyst tubes accommodated in the shell and tube vessel appropriately runs to at least 1000, preferably to at least 5000. Frequently, the number of catalyst tubes accommodated in the reaction vessel is 15 000 to 40 000. Shell and tube reactors having a number of catalyst tubes exceeding 45 000 are usually exceptional. Within the vessel, the catalyst tubes are normally arranged in homogeneous distribution, the distribution appropriately being selected such that the separation of the central internal axes of mutually adjacent catalyst tubes (called the catalyst tube pitch) is 35 to 45 mm (cf. EP 0 468 290 B1).

The partial oxidation can alternatively also be conducted in a multizone (for example “two-zone”) multiple catalyst tube fixed bed reactor as recommended by DE 199 10 506 A1, DE 103 13 213 A1, DE 103 13 208 A1 and EP 1 106 598 A2, particularly at elevated space velocities of the organic compound to be partially oxidized on the catalyst charge of the multiple catalyst tube reactor. A typical catalyst tube length in the case of a two-zone, multiple catalyst tube fixed bed reactor is, for example, 3.50 m, 4.00 m or 4.50 m. Everything else is essentially as described for the one-zone multiple catalyst tube fixed bed reactor. In each temperature control zone (the one-zone multiple catalyst tube reactor fixed bed reactor has just one temperature control zone) of the one-zone or multizone multiple catalyst tube fixed bed reactor, a heat exchange medium is conducted around the catalyst tubes within which the catalyst charge (the fixed catalyst bed) is present. Suitable examples of heat exchange media are melts of salts such as potassium nitrate, potassium nitrite, sodium nitrite and/or sodium nitrate, or of low-melting metals such as sodium, mercury and alloys of different metals. The flow rate of the heat exchange medium within the respective temperature control zone is generally chosen such that the temperature of the heat exchange medium rises from the entry point into the temperature control zone to the exit point from the temperature control zone by 0 to 15 °C, frequently 1 to 10 °C, or 2 to 8 °C, or 3 to 6 °C.

The inlet temperature of the heat exchange medium which, viewed over the respective temperature control zone, can be guided in cocurrent or countercurrent to the reaction gas mixture is preferably chosen as recommended in documents EP 1 106 598 A2, DE 199 48 523 A1, DE 199 48 248 A1, DE 103 13 209 A1, EP 0 700 714 A1, DE 103 13 208 A1, DE 103 13 213 A1, WO 00/53557, WO 00/53558, WO 01/36364, WO 00/53557 and the other documents cited as prior art in those documents. Within the temperature control zone, the heat exchange medium is preferably guided in a meandering manner. The difference between the highest and lowest temperatures of the heat exchange medium within a temperature control zone should, considered over that length section of the temperature control zone in which there is a catalytically active section (not a pure inert bed) of the fixed catalyst bed, advantageously be ≥0 °C and ≤5 °C (this difference is preferably small). In general, the multiple catalyst tube fixed bed reactor additionally has thermal tubes for determination of the temperature of the reaction gas in the catalyst bed (thermal tubes and reaction tubes are charged with the same fixed bed). In an appropriate manner, the internal diameter of the thermal tubes and the diameter of the receiving well (thermowell) for the thermocouple which is centered within it and runs parallel to the longitudinal axis of the thermal tube is chosen such that the ratio of volume that evolves heat of reaction to heat-removing surface area in thermal tubes and working tubes is the same or only slightly different.

The pressure drop in the case of working tubes and thermal tubes should be the same, based on the same GHSV (=strength of the volume flow rate of the reaction gas mixture entering the tube divided by the bed volume of the fixed catalyst bed present in the tube). Compensation of pressure drop in a thermal tube can be effected, for example, by adding catalyst spall to the shaped unsupported catalyst bodies. This compensation is appropriately effected homogeneously over the entire thermal tube length. For the rest, the filling of the thermal tubes can be configured as described in EP 0 873 783 A1.

For provision of the catalyst charge in the catalyst tubes, as already mentioned, it is possible to use solely shaped unsupported catalyst bodies (for example in annular form) that are obtainable as described, or else, for example, largely homogeneous mixtures of shaped unsupported catalyst bodies (for example in annular form) that are obtainable as described and shaped bodies that do not include active composition and are essentially inert with regard to the heterogeneously catalyzed partial gas phase oxidation. Useful materials for such inert shaped bodies include, for example, porous or nonporous aluminum oxides, silicon dioxide, zirconium dioxide, silicon carbide, silicates such as magnesium or aluminum silicate and/or steatite (for example of the C220 type from CeramTec, Germany).

The geometry of such inert shaped diluent bodies is as desired. This means that they may, for example, be spheres, polygons, solid cylinders or else, as in the case of annular shaped catalyst bodies for example, rings. Frequently, the inert shaped diluent bodies chosen will be those whose geometry corresponds to that of the shaped catalyst bodies to be diluted therewith. Along the catalyst charge, however, the geometry of the shaped catalyst body may also be changed or shaped catalyst bodies of different geometry may be used in a largely homogeneous blend. In a less preferred procedure, the active composition of the shaped catalyst body may also be altered along the catalyst charge.

Quite generally, the catalyst charge is advantageously configured such that the volume-specific activity (i.e. that normalized to the unit of volume) either remains constant or increases (continuously, abruptly or in stages) in flow direction of the reaction gas mixture.

A reduction in the volume-specific activity can be achieved in a simple manner, for example, by homogeneously diluting a basic amount of shaped unsupported catalyst bodies (for example in annular form) that have been produced uniformly in accordance with the invention with inert shaped diluent bodies. The higher the proportion of shaped diluent bodies chosen, the smaller the amount of active composition or catalyst activity present in a particular volume of the charge. A reduction can also be achieved by altering the geometry of the shaped unsupported catalyst bodies obtainable in accordance with the invention in such a way that the amount of active composition present in the unit of the inner reaction tube volume becomes smaller.

For the heterogeneously catalyzed gas phase partial oxidations with shaped unsupported catalyst bodies (for example in annular form) that are obtainable as described, the catalyst charge is preferably either configured over its entire length homogeneously with just one type of shaped unsupported catalyst bodies or configured as follows. At the reactor inlet, an essentially homogeneous mixture of shaped unsupported catalyst bodies (for example in annular form) and inert shaped diluent bodies (both preferably having essentially the same geometry) is positioned over a length of 10% to 60%, preferably 10% to 50%, more preferably 20% to 40% and most preferably 25% to 35% (i.e., for example, at a length of 0.70 to 1.50 m, preferably 0.90 to 1.20 m), in each case of the total length of the catalyst charge, where the proportion by weight of the shaped diluent bodies (the bulk densities of shaped catalyst bodies and of shaped diluent bodies generally differ only slightly) is normally 5% to 40% by weight, or 10% of 40% by weight, or 20% to 40% by weight, or 25% to 35% by weight. This first charge section is then advantageously followed, up to the end of the length of the catalyst charge (i.e., for example, over a length of 1.00 to 3.00 m or of 1.00 to 2.70 m, preferably 1.40 to 3.00 m, or 2.00 to 3.00 m), either by a bed of the same shaped unsupported catalyst bodies (for example in annular form) obtainable as described that has been diluted merely to a lesser degree (than in the first section), or, most preferably, a 100% (undiluted) bed of the same shaped unsupported catalyst bodies (for example in annular form) that has also been used in the first section. It is of course also possible to choose a constant dilution over the entire charge. It is also possible to charge the first section merely with a shaped unsupported catalyst body (for example annular shaped unsupported catalyst body) of low active composition density based on its occupation of space, and the second section with a shaped unsupported catalyst body (for example annular shaped unsupported catalyst body) obtainable in accordance with the invention with a high active composition density based on its occupation of space (for example 6.5 mm×3 mm×4.5 mm [external diameter×height×internal diameter] in the first section, and 5×2×2 mm [E×H×I] in the second section).

In flow direction of the reaction gas mixture upstream of the actual fixed bed catalyst charge, a bed of inert shaped bodies may be provided, which pursues the purpose, for example, of increasing the inlet temperature of the reaction gas mixture to the temperature of the heat exchange medium.

Moreover, according to the teachings of the prior art (for example WO 2012/049246), the catalyst charge (especially the fresh catalyst charge) of the reactor and the process conditions for the catalyst partial oxidation, in the case of the heterogeneously catalyzed partial oxidation of propene to acrolein (or in the case of a heterogeneously catalyzed partial oxidation for preparation of methacrolein), are preferably chosen (configured) such that, in partial oxidation operation, there is no point in the fixed catalyst bed at which the difference between the fixed catalyst bed temperature at this point and the temperature of the heat exchange medium at the level of this point is ≥120 °C. Advantageously, however, this temperature difference at every point, in the positive direction, is ≤100 °C (especially 40 to 100 °C), particularly advantageously ≤90 °C (especially 50 to 90 °C). In principle, however, this (positive) temperature difference may also be ≤50 °C or ≤40 °C. Moreover, the configuration is preferably such that this temperature difference, in the event of an increase in the temperature of the heat exchange medium by 1 °C, rises at any point in the fixed catalyst bed by >0 °C but ≤+9 °C, better ≤+7 °C, preferably ≤+5 °C, more preferably ≤+3 °C (see also EP 1 106 598 A1).

Otherwise, a heterogeneously catalyzed partial oxidation for preparation of acrolein (from propene) or from methacrolein (from the C₄ precursor compounds mentioned in the present document) with annular shaped unsupported catalyst bodies produced in accordance with the invention can advantageously be executed in an entirely corresponding manner to the details in WO 2015/067656.

Overall, in the case of a partial oxidation for preparation of acrolein or methacrolein which is conducted with the shaped unsupported catalyst bodies (for example in annular form) that are obtainable as described as catalysts, the catalyst charge, the starting reaction gas mixture, the space velocity and the reaction temperature are generally chosen so as to result in, in a single pass of the reaction gas mixture through the catalyst charge, a conversion of the organic compound to be partially oxidized (propene, isobutene, tert-butanol or the methyl ether thereof) of at least 90 mol %, or at least 92 mol %, preferably of at least 93 mol % or at least 94 mol %, or at least 95 mol % or at least 97 mol %, but normally ≤99 mol %. The selectivity for the products of value (total of acrolein and acrylic acid or total of methacrolein and methacrylic acid) here will regularly be ≥80 mol %, or ≥85 mol % or ≥90 mol %.

Finally, it should be emphasized that shaped unsupported catalyst bodies of the invention that are obtainable as described also have advantageous fracture characteristics in reactor charging.

The startup of a fresh catalyst charge (fixed catalyst bed) comprising shaped unsupported catalyst bodies (for example in annular form) that are obtainable in accordance with the invention can be effected, for example, as described in DE 103 37 788 A1 or in DE 10 2009 047291 A1.

The forming of shaped unsupported catalyst bodies obtainable in accordance with the invention can be accelerated by conducting it at essentially homogeneous conversion at elevated space velocity of reaction gas input mixture on the catalyst charge.

Otherwise, unsupported catalysts obtainable in accordance with the invention are quite generally suitable for catalysis of the gas phase partial (amm)oxidation of an alkanol, alkanal, alkene, alkane and alkenal having 3 to 6 (i.e. 3, 4, 5 or 6) carbon atoms to olefinically unsaturated aldehydes and/or carboxylic acids, for example, and the corresponding nitriles, and for oxidative dehydrogenations on the gas phase catalysis of the aforementioned organic compounds comprising 3, 4, 5 or 6 carbon atoms.

Industrial scale production of shaped unsupported catalyst bodies of the invention is appropriately effected analogously to the manner described in DE 10 2008 040093 A1 and DE 10 2008 040094 A1 (particularly advantageously as in the illustrative embodiments of these documents).

Thus, the present application especially encompasses the following embodiments of the invention:

Thus, the present invention especially encompasses the following embodiments of the invention:
1. A process for producing shaped unsupported catalyst bodies for gas phase oxidation of an alkene and/or an alcohol to an α, β-unsaturated aldehyde and/or an α, β-unsaturated carboxylic acid, where the shaped unsupported catalyst bodies comprise at least the elements molybdenum, bismuth, iron and cobalt, where
a) at least one source of the elemental constituents molybdenum, bismuth, iron, cobalt and optionally at least one source of the elemental constituent nickel is used in each case to produce an aqueous solution or aqueous suspension,
b) a powder P is produced by drying the aqueous solution or aqueous suspension obtained in a) and optionally comminuting,
c) the powder P obtained in b), optionally with addition of one or more auxiliaries and after uniform mixing and optional compaction, is compacted to shaped precursor bodies having a cylindrical structure, and
d) the shaped precursor bodies obtained in c) are subjected to thermal treatment to form the shaped unsupported catalyst bodies,
wherein the pressure in the compaction in c) is chosen such that the density of the shaped precursor bodies is from 1.70 to 2.30 g/cm³, where the density of the shaped precursor bodies is the quotient of the mass and geometric volume, and the weight loss in the thermal treatment in d) is from 25% to 40% by weight.
2. The process according to embodiment 1, wherein the pressure in the compaction in c) is chosen such that the density of the shaped precursor bodies is from 1.72 to 2.28 g/cm³.
3. The process according to embodiment 1 or 2, wherein the pressure in the compaction in c) is chosen such that the density of the shaped precursor bodies is from 1.74 to 2.26 g/cm³.
4. The process according to any of embodiments 1 to 3, wherein the pressure in the compaction in c) is chosen such that the density of the shaped precursor bodies is from 1.76 to 2.24 g/cm³.
5. The process according to any of embodiments 1 to 4, wherein the pressure in the compaction in c) is chosen such that the density of the shaped precursor bodies is from 1.78 to 2.22 g/cm³.
6. The process according to any of embodiments 1 to 5, wherein the pressure in the compaction in c) is chosen such that the density of the shaped precursor bodies is from 1.80 to 2.20 g/cm³.
7. The process according to any of embodiments 1 to 6, wherein the weight loss in the thermal treatment in d) is from 26% to 39% by weight.
8. The process according to any of embodiments 1 to 7, wherein the weight loss in the thermal treatment in d) is from 27% to 38% by weight.
9. The process according to any of embodiments 1 to 8, wherein the weight loss in the thermal treatment in d) is from 28% to 38% by weight.
10. The process according to any of embodiments 1 to 9, wherein the weight loss in the thermal treatment in d) is from 29% to 37% by weight.
11. The process according to any of embodiments 1 to 10, wherein the weight loss in the thermal treatment in d) is from 30% to 35% by weight.
12. The process according to any of embodiments 1 to 11, wherein the cylindrical structure is
i) a cylindrical body having a central circular aperture in the longitudinal direction or
ii) a cylindrical body having three uniform notches in the longitudinal direction and having three circular apertures arranged uniformly in the longitudinal direction.
13. The process according to any of embodiments 1 to 12, wherein the cylindrical structure is a hollow cylinder.
14. The process according to any of embodiments 1 to 13, wherein the molybdenum content of the shaped unsupported catalyst bodies, calculated as MoO₃, is from 45% to 75% by weight.
15. The process according to any of embodiments 1 to 14, wherein the molybdenum content of the shaped unsupported catalyst bodies, calculated as MoO₃, is from 50% to 70% by weight.
16. The process according to any of embodiments 1 to 15, wherein the molybdenum content of the shaped unsupported catalyst bodies, calculated as MoO₃, is from 55% to 65% by weight.
17. The process according to any of embodiments 1 to 16, wherein the bismuth content of the shaped unsupported catalyst bodies, calculated as Bi₂O₃, is from 1% to 20% by weight.
18. The process according to any of embodiments 1 to 17, wherein the bismuth content of the shaped unsupported catalyst bodies, calculated as Bi₂O₃, is from 2% to 15% by weight.
19. The process according to any of embodiments 1 to 18, wherein the bismuth content of the shaped unsupported catalyst bodies, calculated as Bi₂O₃, is from 3% to 10% by weight.
20. The process according to any of embodiments 1 to 19, wherein the iron content of the shaped unsupported catalyst bodies, calculated as Fe₂O₃, is from 2% to 12% by weight.
21. The process according to any of embodiments 1 to 20, wherein the iron content of the shaped unsupported catalyst bodies, calculated as Fe₂O₃, is from 3% to 11% by weight.
22. The process according to any of embodiments 1 to 21, wherein the iron content of the shaped unsupported catalyst bodies, calculated as Fe₂O₃, is from 4% to 10% by weight.
23. The process according to any of embodiments 1 to 22, wherein the total cobalt and nickel content of the shaped unsupported catalyst bodies, calculated as CoO and NiO, is from 9% to 30% by weight.
24. The process according to any of embodiments 1 to 23, wherein the total cobalt and nickel content of the shaped unsupported catalyst bodies, calculated as CoO and NiO, is from 12% to 27% by weight.
25. The process according to any of embodiments 1 to 24, wherein the total cobalt and nickel content of the shaped unsupported catalyst bodies, calculated as CoO and NiO, is from 15% to 24% by weight.
26. The process according to any of embodiments 1 to 25, wherein the shaped unsupported catalyst bodies additionally comprise the elements potassium and/or silicon.
27. A shaped unsupported catalyst body having a cylindrical structure for gas phase oxidation of an alkene and/or an alcohol to an α, β-unsaturated aldehyde and/or an α, β-unsaturated carboxylic acid, obtainable by a process of embodiments 1 to 26, where the shaped unsupported catalyst body comprises at least the elements molybdenum, bismuth, iron and cobalt, where the density of the shaped unsupported catalyst body is from 1.20 to 1.70 g/cm³, where the density of the shaped unsupported catalyst body is the quotient of the mass and geometric volume, the total pore volume of the shaped unsupported catalyst body is from 0.33 to 0.60 cm³/g and the pore volume of the shaped unsupported catalyst body in the range from 0.1 to 1 μm is from 85% to 99% of the total pore volume, where the total pore volume and the pore volume in the range from 0.1 to 1 μm is determined by mercury porosimetry.
28. The shaped unsupported catalyst body according to embodiment 27, wherein the density of the shaped unsupported catalyst body is from 1.22 to 1.68 g/cm³.
29. The shaped unsupported catalyst body according to embodiment 27 or 28, wherein the density of the shaped unsupported catalyst body is from 1.24 to 1.66 g/cm³.
30. The shaped unsupported catalyst body according to any of embodiments 27 to 29, wherein the density of the shaped unsupported catalyst body is from 1.26 to 1.64 g/cm³.
31. The shaped unsupported catalyst body according to any of embodiments 27 to 30, wherein the density of the shaped unsupported catalyst body is from 1.28 to 1.62 g/cm³.
32. The shaped unsupported catalyst body according to any of embodiments 27 to 31, wherein the density of the shaped unsupported catalyst body is from 1.30 to 1.60 g/cm³.
33. The shaped unsupported catalyst body according to any of embodiments 27 to 32, wherein the total pore volume of the shaped unsupported catalyst body is from 0.34 to 0.58 cm³/g.
34. The shaped unsupported catalyst body according to any of embodiments 27 to 33, wherein the total pore volume of the shaped unsupported catalyst body is from 0.35 to 0.56 cm³/g.
35. The shaped unsupported catalyst body according to any of embodiments 27 to 34, wherein the total pore volume of the shaped unsupported catalyst body is from 0.36 to 0.54 cm³/g.
36. The shaped unsupported catalyst body according to any of embodiments 27 to 35, wherein the total pore volume of the shaped unsupported catalyst body is from 0.37 to 0.52 cm³/g.
37. The shaped unsupported catalyst body according to any of embodiments 27 to 36, wherein the total pore volume of the shaped unsupported catalyst body is from 0.38 to 0.50 cm³/g.
38. The shaped unsupported catalyst body according to any of embodiments 27 to 37, wherein the pore volume of the shaped unsupported catalyst body in the range from 0.1 to 1 μm is from 86% to 98% of the total pore volume.
39. The shaped unsupported catalyst body according to any of embodiments 27 to 38, wherein the pore volume of the shaped unsupported catalyst body in the range from 0.1 to 1 μm is from 87% to 97% of the total pore volume.
40. The shaped unsupported catalyst body according to any of embodiments 27 to 39, wherein the pore volume of the shaped unsupported catalyst body in the range from 0.1 to 1 μm is from 88% to 96% of the total pore volume.
41. The shaped unsupported catalyst body according to any of embodiments 27 to 40, wherein the pore volume of the shaped unsupported catalyst body in the range from 0.1 to 1 μm is from 89% to 95% of the total pore volume.
42. The shaped unsupported catalyst body according to any of embodiments 27 to 41, wherein the cylindrical structure is
i) a cylindrical body having a central circular aperture in the longitudinal direction or
ii) a cylindrical body having three uniform notches in the longitudinal direction and having three circular apertures arranged uniformly in the longitudinal direction.
43. The shaped unsupported catalyst body according to embodiment 42, wherein the shortest distance between the outer wall of the cylindrical body and the next aperture is from 0.75 to 2.5 mm.
44. The shaped unsupported catalyst body according to embodiment 42 or 43, wherein the shortest distance between the outer wall of the cylindrical body and the next aperture is from 0.8 to 2.0 mm.
45. The shaped unsupported catalyst body according to any of embodiments 42 to 44, wherein the shortest distance between the outer wall of the cylindrical body and the next aperture is from 1.0 to 1.8 mm.
46. The shaped unsupported catalyst body according to any of embodiments 42 to 45, wherein the shortest distance between the outer wall of the cylindrical body and the next aperture is from 1.2 to 1.7 mm.
47. The shaped unsupported catalyst body according to any of embodiments 42 to 46, wherein the shortest distance between the outer wall of the cylindrical body and the next aperture is from 1.3 to 1.6 mm.
48. The shaped unsupported catalyst body according to any of embodiments 27 to 42, wherein the cylindrical structure is a hollow cylinder.
49. The shaped unsupported catalyst body according to embodiment 48, wherein the wall thickness of the hollow cylinder is from 0.75 to 2.5 mm.
50. The shaped unsupported catalyst body according to embodiment 48 or 49, wherein the wall thickness of the hollow cylinder is from 0.8 to 2.0 mm.
51. The shaped unsupported catalyst body according to any of embodiments 48 to 50, wherein the wall thickness of the hollow cylinder is from 1.0 to 1.8 mm.
52. The shaped unsupported catalyst body according to any of embodiments 48 to 51, wherein the wall thickness of the hollow cylinder is from 1.2 to 1.7 mm.
53. The shaped unsupported catalyst body according to any of embodiments 48 to 52, wherein the wall thickness of the hollow cylinder is from 1.3 to 1.6 mm.
54. The shaped unsupported catalyst body according to any of embodiments 27 to 53, wherein the shaped unsupported catalyst body is a multielement oxide of the general formula I

Mo₁₂BiₐFe_{b}Co_{c}Ni_{d}XₑY_{f}Z_{g}Oₙ (1)
with
X=K, Cs and/or Rb
Y=Ca, Sr, Ba, Li, Na, Cr, W, Mn, Cu, Zn, Ga, P, B, As, Sn, Sb, Te, Nb, Ta, Pb, Ce and/or La
Z=Si, Al, Ti, Zr and/or Mg
a=0.2 to 2
b=1 to 4
c=3 to 9
d=0 to 4
c+d=4 to 9.5
e=0.01 to 0.5
f=0 to 10
g=0 to 10
n=a number which is determined by the valency and frequency of the elements in the general formula I other than oxygen.
55. A process for preparing an α, β-unsaturated aldehyde and/or an α, β-unsaturated carboxylic acid, wherein an alkene and/or an alcohol is guided together with molecular oxygen over a fixed catalyst bed comprising a bed of shaped unsupported catalyst bodies according to any of embodiments 27 to 54.
56. The process according to embodiment 55, wherein the propene is used as alkene.
57. A fixed bed reactor comprising a bed of shaped unsupported catalyst bodies according to any of embodiments 27 to 55.

### EXAMPLES

Production of shaped unsupported catalyst bodies K1 to K10:

### Example 1

Production of shaped unsupported catalyst precursor body K1, where the active multimetal oxide had the stoichiometry Mo₁₂Co₇Fe₃Bi_{0.6}Si_{1.6}K_{0.08}Oₓ.

The stirred vessels used were each filled in the presence of ambient air. In the course of stirring/mixing, they were sealed airtight with a lid that had a pressure release valve to the atmosphere (1.01 bar).

### a) Preparation of an Aqueous Solution B

A temperature-controllable stainless steel (EN 1.4541) cylindrical stirred vessel (capacity 1.75 m³, diameter 1.3 m) equipped with a stirrer was initially charged with 430 kg of demineralized water and heated to 60 °C while stirring (70 rpm).

Subsequently, with constant stirring and while maintaining the temperature of 60 °C, 0.61 kg of an aqueous potassium hydroxide solution (47.5% by weight of KOH) that had a temperature of 20 °C was added within one minute. While maintaining the 60 °C, with constant stirring, 136.2 kg of fine granular ammonium heptamolybdate tetrahydrate (54.3% by weight of Mo, supplier: NIPPON INORGANIC COLOUR & CHEMICAL CO., LTD., 3-14-1 Funado Itabashi-ku Tokyo 174-0041 Japan) at a temperature of 25 °C was then stirred in in portions, and the resulting aqueous solution (which was slightly turbid as a result of a tiny amount of insoluble isopolymolybdate contaminants) was stirred (70 rpm) at 60 °C for a further 60 minutes.

### b) Preparation of an Aqueous Solution A

A temperature-controllable cylindrical stainless steel (EN 1.4541) stirred vessel (capacity 1.75 m³, diameter 1.3 m) equipped with a stirrer was initially charged with 212 kg of an aqueous cobalt(II) nitrate solution in nitric acid (12.5% by weight of Co, 27% by weight of nitrate, prepared by dissolving cobalt metal from MFT Metals % Ferro-Alloys Trading GmbH, D-41474 Viersen, purity >99.6% by weight of Co, <0.3% by weight of Ni, <100 mg/kg Fe, <50 mg/kg Cu in aqueous nitric acid), which was heated to 60 °C while stirring (70 rpm).

With continued stirring (70 rpm) and continued heating to 60 °C, 78 kg of an iron(III) nitrate nonahydrate melt at 60 °C (13.8% by weight of Fe, <0.4% by weight of alkali metals, <0.01% by weight of chloride, <0.02% by weight of sulfate, from Dr. Paul Lohmann GmbH, D-81857 Emmerthal) was metered in, and the mixture was stirred (70 rpm) at 60 °C for a further 30 minutes.

While maintaining the 60 °C and with continued stirring (70 rpm), 72.6 kg of an aqueous bismuth nitrate solution in nitric acid at 60 °C (11.1% by weight of Bi, 13% by weight of nitrate, prepared by dissolving bismuth metal from Sidech S.A., BE-1495 Tilly, purity >99.997% by weight of Bi, <7 mg/kg Pb, <5 mg/kg each of Ni, Ag, Fe, <3 mg/kg each of Cu, Sb, and <1 mg/kg each of Cd, Zn, in aqueous nitric acid) was added to the resulting aqueous solution, and the mixture was then stirred (70 rpm) at 60 °C for another 30 minutes.

c) Mixing of Aqueous Solution A with Aqueous Solution B

Aqueous solution A at 60 °C was metered continuously into the vigorously stirred (70 rpm) aqueous solution B that had been kept at 60 °C within 15 minutes. The resultant aqueous suspension was stirred at 60 °C for another 15 minutes.

### d) Addition of a Silica Sol to Obtain an Aqueous Mixture M

Immediately after the further stirring had ended, 12.6 kg of silica gel from Grace GmbH & KG, In der Hollerecke 1, D-67545 Worms, of the LUDOX TM50 type (47.5% by weight of SiO₂), that had been heated to 60 °C was then added to the aqueous mixture obtained in c).

### e) Spray Drying of the Aqueous Mixture M

The aqueous mixture M was spray-dried immediately after its production. The aqueous mixture (suspension) M, stirring of which was continued in each case (even during spray drying) at 60 °C by means of the stirrer (40 rpm), was spray-dried in a spray tower (model S-50-N/R from Niro A/S, made of 1.4541 stainless steel) having a centrifugal atomizer of the FS-15 type and an atomizer wheel of the AM8-150 type in a cocurrent hot air flow (gas inlet temperature: 350±10 °C, gas outlet temperature: 140±5 °C, gas flow rate: 2200±100 m³ (STP)/h, speed of the atomizer wheel: 20 000 rpm).

The resultant spray powder was stored intermediately until further processing in airtight containers (capacity 200 L or 1000 L, 25 °C, atmospheric pressure) (for 10 calendar days; shorter or longer intermediate storage (for up to 30 calendar days) had no effect on the resulting outcomes). The ignition loss of the spray powder obtained (calcine at 600 °C (powder temperature) under stationary air in excess for 3 h) was 31±2% by weight of its starting weight. The spray powder had a D50 of 35±10 μm (D50 means that 50% of the particles are smaller than the value reported) at dispersion pressure of 2.0 bar absolute.

### f) Production of Ringlike Shaped Precursor Bodies

100 kg of spray powder and 1 kg of graphite (3160 type from Asbury Graphite Mills Inc., New Jersey 08802) were then initially charged in a slanting mixer (model: VIL, capacity: 200 I, Aachener Misch- und Knetmaschinenfabrik) having mixing and cutting blades (mixing blade speed: 39 rpm, cutting blade speed: 3000 rpm) and premixed for 5 minutes.

The resulting mixture was then compacted in a K200/100 compactor from Hosokawa Bepex GmbH with concave, fluted smooth rollers (roller diameter 100 mm, roller length 200 mm, gap width: 2.8 mm, roller speed: 5 rpm, target compression force: 80 kN, length-specific target compression force 8 kN/cm). Integrated vibrating screens from Allgaier (oversize screen size: 1.5 mm, undersize screen size: 200 μm) with ball-type screening aids (diameter 22 mm) were used to isolate a compactate having a particle size for the most part between 200 μm and 1.5 mm.

For the tableting, a further 2.5% by weight of the graphite (3160 type from Asbury Graphite Mills Inc., New Jersey 08802) was added to the compactate in a turbulent mixer from Drais over the course of 2 minutes.

Subsequently, the granular material obtained was compacted (tableted) with the aid of a Korsch XT-600 rotary press (with 65 EURO B dies) under a dry air atmosphere to give ringlike shaped precursor bodies of geometry E×H×I=5 mm×5 mm×2 mm with an uncurved (i.e. planar) end face of mass (Mᵥ) 188 mg. The initial compressive force applied (compression force) was 0.7 kN; the main compressive force applied (compression force) was 2.7 kN. The speed of the rotor was 40 rpm. The side crushing strength (SDFᵥ) of the resulting ringlike shaped precursor bodies was 23 N.

### g) Thermal Pretreatment and Calcination of the Ringlike Shaped Precursor Bodies Produced in f)

The shaped rings were placed onto the belt of a belt calcining apparatus (as described in WO 2002/024620) having eight chambers (chamber width 100 cm, chamber length 150 cm).

Chambers 1 to 8 each had a ventilator to generate air circulation and were thermostatted at 150 °C, 190 °C, 220 °C, 265 °C, 380 °C, 430 °C, 520 °C and 520 °C. Heated air was metered into each chamber. The air feed rate to the respective chambers 1 to 8 was 90 m³ (STP)/h, 130 m³ (STP)/h, 255 m³ (STP)/h, 90 m³ (STP)/h, 150 m³ (STP)/h, 90 m³ (STP)/h, 90 m³ (STP)/h and 140 m³ (STP)/h. Waste air was removed from each chamber by means of a ventilator. The air removal rate from the respective chambers 1 to 8 was 130 m³ (STP)/h, 290 m³ (STP)/h, 305 m³ (STP)/h, 144 m³ (STP)/h, 135 m³ (STP)/h, 80 m³ (STP)/h, 80 m³ (STP)/h and 130 m³ (STP)/h.

The bed height of chambers 1 to 4 was 40 mm. The bed height of chambers 5 to 8 was 75 mm. Within the chambers, the temporal and local variance of the temperature from the target value was always ≤10 °C.

The belt speed was such that the residence time in each of the first four chambers was 105 minutes, and in each of the fifth to eighth chambers 270 minutes.

In this way, 2.6 t of shaped unsupported catalyst bodies K1 was produced.

### Example 2 (not According to the Invention)

Production of shaped unsupported catalyst body K2, where the active multimetal oxide had the stoichiometry Mo₁₂Co₇Fe₃Bi_{0.6}Si_{1.6}K_{0.08}Oₓ.

Shaped unsupported catalyst bodies K2 were produced in the same way as shaped unsupported catalyst body K1, with a modification in f).

The shaped precursor bodies were compacted (tableted) to a mass (Mᵥ) of 220 mg. The initial compressive force applied (compression force) was 1.3 kN; the main compressive force applied (compression force) was 6.8 kN. The side crushing strength (SDFᵥ) of the resulting ringlike shaped precursor bodies was 48 N.

### Example 3

Production of shaped unsupported catalyst body K3, where the active multimetal oxide had the stoichiometry Mo₁₂Co₇Fe₃Bi_{0.6}Si_{1.6}K_{0.08}Oₓ.

The shaped unsupported catalyst bodies K3 were produced in the same way as the shaped unsupported catalyst body K1, with modifications in f) and g).

The granular material obtained was compacted (tableted) in f) with the aid of a Kilian E150+ rotary press (with 21 EURO D dies) under a dry air atmosphere to give ringlike shaped precursor bodies of geometry E×H×I=5 mm×3 mm×2 mm with an uncurved (i.e. planar) end face of mass (Mᵥ) 90 mg. The speed of the rotor was 25 rpm. The side crushing strength (SDFᵥ) of the resulting ringlike shaped precursor bodies was 6 N.

1000 g of the shaped precursor bodies produced in f), divided equally between four mesh grids arranged alongside one another and each having a base area of 150 mm×150 mm (bed height: 15 mm), was applied in an air circulation shaft oven (from Nabertherm GmbH, D-28865 Lilienthal; oven model: S60/65A) with a flow of 4500 L (STP)/h of predried air (at an inlet temperature of 140 °C) (the air circulation oven was in an environment at 25 °C). Subsequently, while maintaining the air flow (including its inlet temperature), the temperature in the air circulation shaft oven was varied as follows (the temperature FIGURES mean the temperature in the bulk material applied in each case; these were determined by means of four thermocouples, each of which was in the geometric middle of the four mesh grids in the center of the bulk material applied to the respective mesh grid; one of the thermocouples provided the actual value for temperature control of the air circulation shaft oven; the other thermocouples confirmed that the temperatures were identical within the ±0.1 °C interval). The temperature increases were essentially linear over time. Heating was effected from 25 °C to 130 °C within 72 minutes. The temperature was kept at 130 °C for 72 minutes before being increased to 190 °C within 36 minutes. The temperature was kept at 190 °C for 72 minutes before being increased to 220 °C within 36 minutes. The temperature was kept at 220 °C for 72 minutes before being increased to 265 °C within 36 minutes. The temperature was kept at 265 °C for 72 minutes before being increased to 380 °C within 93 minutes. The temperature was kept at 380 °C for 187 minutes before being increased to 430 °C within 93 minutes. The temperature was kept at 430 °C for 187 minutes before being increased to the final calcination temperature of 500 °C within 93 minutes. This was maintained for 463 minutes. Then cooling was effected to 25 °C within 12 hours. For this purpose, both the heating of the air circulation shaft oven and the air stream preheating were switched off (however, the air stream of 4500 L (STP)/h itself was maintained; the inlet temperature of the air stream was then 25 °C).

### Example 4 (not According to the Invention)

Production of shaped unsupported catalyst body K4, where the active multimetal oxide had the stoichiometry Mo₁₂Co₇Fe₃Bi_{0.6}Si_{1.6}K_{0.08}Oₓ.

Shaped unsupported catalyst bodies K4 were produced in the same way as shaped unsupported catalyst body K3, with a modification in f).

The shaped precursor bodies were compacted (tableted) to a mass (Mᵥ) of 122 mg. The side crushing strength (SDFᵥ) of the resulting ringlike shaped precursor bodies was 22 N.

### Example 5

Production of shaped unsupported catalyst body K5, where the active multimetal oxide had the stoichiometry Mo₁₂Co_{8.3}Fe_{2.1}Bi_{0.6}Si_{1.6}K_{0.08}Oₓ.

The stirred vessels used were each filled in the presence of ambient air. In the course of stirring/mixing, they were sealed airtight with a lid that had a pressure release valve to the atmosphere (1.01 bar).

### a) Preparation of an Aqueous Solution B

A temperature-controllable stainless steel (EN 1.4541) cylindrical stirred vessel (capacity 1.75 m³, diameter 1.3 m) equipped with a stirrer was initially charged with 654 kg of demineralized water and heated to 60 °C while stirring (70 rpm).

Subsequently, with constant stirring and while maintaining the temperature of 60 °C, 0.36 kg of an aqueous potassium hydroxide solution (47.5% by weight of KOH) that had a temperature of 20 °C was added within one minute. While maintaining the 60 °C, with constant stirring, 80.4 kg of fine granular ammonium heptamolybdate tetrahydrate (54.3% by weight of Mo, supplier: NIPPON INORGANIC COLOUR & CHEMICAL CO., LTD., 3-14-1 Funado Itabashi-ku Tokyo 174-0041 Japan) at a temperature of 25 °C was then stirred in in portions, and the resulting aqueous solution (which was slightly turbid as a result of a tiny amount of insoluble isopolymolybdate contaminants) was stirred (70 rpm) at 60 °C for a further 60 minutes.

### b) Preparation of an Aqueous Solution A

A temperature-controllable cylindrical stainless steel (EN 1.4541) stirred vessel (capacity 1.75 m³, diameter 1.3 m) equipped with a stirrer was initially charged with 148.4 kg of an aqueous cobalt(II) nitrate solution in nitric acid (12.5% by weight of Co, 27% by weight of nitrate, prepared by dissolving cobalt metal from MFT Metals % Ferro-Alloys Trading GmbH, D-41474 Viersen, purity >99.6% by weight of Co, <0.3% by weight of Ni, <100 mg/kg Fe, <50 mg/kg Cu in aqueous nitric acid), which was heated to 60 °C while stirring (70 rpm).

With continued stirring (70 rpm) and continued heating to 60 °C, 32.3 kg of an iron(III) nitrate nonahydrate melt at 60 °C (13.8% by weight of Fe, <0.4% by weight of alkali metals, <0.01% by weight of chloride, <0.02% by weight of sulfate, from Dr. Paul Lohmann GmbH, D-81857 Emmerthal) was metered in, and the mixture was stirred (70 rpm) at 60 °C for a further 30 minutes.

While maintaining the 60 °C and with continued stirring (70 rpm), 42.8 kg of an aqueous bismuth nitrate solution in nitric acid at 60 °C (11.1% by weight of Bi, 13% by weight of nitrate (NO₃⁻), prepared by dissolving bismuth metal from Sidech S.A., BE-1495 Tilly, purity >99.997% by weight of Bi, <7 mg/kg Pb, <5 mg/kg each of Ni, Ag, Fe, <3 mg/kg each of Cu, Sb, and <1 mg/kg each of Cd, Zn, in aqueous nitric acid) was added to the resulting aqueous solution, and the mixture was then stirred (70 rpm) at 60 °C for another 30 minutes.

c) Mixing of Aqueous Solution A with Aqueous Solution B

Aqueous solution A at 60 °C was metered continuously into the vigorously stirred (70 rpm) aqueous solution B that had been kept at 60 °C within 15 minutes. The resultant aqueous suspension was stirred at 60 °C for another 15 minutes.

### d) Addition of a Silica Sol to Obtain an Aqueous Mixture M

Immediately after the further stirring had ended, 7.4 kg of silica gel from Grace GmbH & KG, In der Hollerecke 1, D-67545 Worms, of the LUDOX TM50 type (47.5% by weight of SiO₂), that had been heated to 60 °C was then added to the aqueous mixture obtained in c).

### e) Spray Drying of the Aqueous Mixture M

The aqueous mixture M was spray-dried immediately after its production. The aqueous mixture (suspension) M, stirring of which was continued in each case (even during spray drying) at 60 °C by means of the stirrer (40 rpm), was spray-dried in a spray tower (model S-50-N/R from Niro A/S, made of 1.4541 stainless steel) in a cocurrent hot air flow (gas inlet temperature: 350±10 °C, gas outlet temperature: 140±5 °C, gas flow rate: 2200±100 m³ (STP)/h, speed of the atomizer wheel: 20 000 rpm).

The resultant spray powder was stored intermediately until further processing in airtight containers (capacity 200 L or 1000 L, 25 °C, atmospheric pressure) (for 10 calendar days; shorter or longer intermediate storage (for up to 30 calendar days) had no effect on the resulting outcomes). The ignition loss of the spray powder obtained (calcine at 600 °C (powder temperature) under stationary air in excess for 3 h) was 31±2% by weight of its starting weight. The spray powder had a D50 of 35±10 μm (D50 means that 50% of the particles are smaller than the value reported) at dispersion pressure of 2.0 bar absolute.

### f) Production of Ringlike Shaped Precursor Bodies

100 kg of spray powder and 1 kg of graphite (3160 type from Asbury Graphite Mills Inc., New Jersey 08802) were then initially charged in a slanting mixer (model: VIL, capacity: 200 I, Aachener Misch- und Knetmaschinenfabrik) having mixing and cutting blades (mixing blade speed: 39 rpm, cutting blade speed: 3000 rpm) and premixed for 5 minutes.

The resulting mixture was then compacted in a K200/100 compactor from Hosokawa Bepex GmbH with concave, fluted smooth rollers (roller diameter 100 mm, roller length 200 mm, gap width: 2.8 mm, roller speed: 5 rpm, target compression force: 80 kN, length-specific target compression force 8 kN/cm). Integrated vibrating screens from Allgaier (oversize screen size: 1.5 mm, undersize screen size: 200 μm) with ball-type screening aids (diameter 22 mm) were used to isolate a compactate having a particle size for the most part between 200 μm and 1.5 mm.

For the tableting, a further 2.5% by weight of the graphite (3160 type from Asbury Graphite Mills Inc., New Jersey 08802) was added to the compactate in a turbulent mixer from Drais over the course of 2 minutes.

Subsequently, the granular material obtained as described was compacted (tableted) with the aid of a Kilian E150+ rotary press (with 21 EURO D dies) under a dry air atmosphere to give ringlike shaped precursor bodies of geometry E×H×I=5 mm×3 mm×2 mm with an uncurved (i.e. planar) end face of mass (Mᵥ) 102 mg. The speed of the rotor was 25 rpm. The side crushing strength (SDFᵥ) of the resulting ringlike shaped precursor bodies was 12 N.

### g) Thermal Pretreatment and Calcination of the Ringlike Shaped Precursor Bodies Produced in f)

1000 g of the shaped precursor bodies produced, divided equally between four mesh grids arranged alongside one another and each having a base area of 150 mm×150 mm (bed height: 15 mm), was applied in an air circulation shaft oven (from Nabertherm GmbH, D-28865 Lilienthal; oven model: S60/65A) with a flow of 4500 L (STP)/h of predried air (at an inlet temperature of 140 °C) (the air circulation oven was in an environment at 25 °C). Subsequently, while maintaining the air flow (including its inlet temperature), the temperature in the air circulation shaft oven was varied as follows (the temperature FIGURES mean the temperature in the bulk material applied in each case; these were determined by means of four thermocouples, each of which was in the geometric middle of the four mesh grids in the center of the bulk material applied to the respective mesh grid; one of the thermocouples provided the actual value for temperature control of the air circulation shaft oven; the other thermocouples confirmed that the temperatures were identical within the ±0.1 °C interval). The temperature increases were essentially linear over time. Heating was effected from 25 °C to 130 °C within 72 minutes. The temperature was maintained at 130 °C for 72 min before being increased to 190 °C within 36 min. The temperature was kept at 190 °C for 72 minutes before being increased to 220 °C within 36 minutes. The temperature was kept at 220 °C for 72 minutes before being increased to 265 °C within 36 minutes. The temperature was kept at 265 °C for 72 minutes before being increased to 380 °C within 93 minutes. The temperature was kept at 380 °C for 187 minutes before being increased to 430 °C within 93 minutes. The temperature was kept at 430 °C for 187 minutes before being increased to the final calcination temperature of 500 °C within 93 minutes. This was maintained for 463 minutes. Then cooling was effected to 25 °C within 12 hours. For this purpose, both the heating of the air circulation shaft oven and the air stream preheating were switched off (however, the air stream of 4500 L (STP)/h itself was maintained; the inlet temperature of the air stream was then 25 °C).

### Example 6 (not According to the Invention)

Production of shaped unsupported catalyst body K6, where the active multimetal oxide had the stoichiometry Mo₁₂Co_{8.3}Fe_{2.1}Bi_{0.6}Si_{1.6}K_{0.08}Oₓ.

Shaped unsupported catalyst bodies K6 were produced in the same way as shaped unsupported catalyst body K5, with a modification in f).

The shaped precursor bodies were compacted (tableted) to a mass (Mᵥ) of 119 mg. The side crushing strength (SDFᵥ) of the resulting ringlike shaped precursor bodies was 26 N.

### Example 7

Production of shaped unsupported catalyst body K7, where the active multimetal oxide had the stoichiometry Mo₁₂Co_{8.3}Fe_{2.1}Bi_{0.6}Si_{1.6}K_{0.08}Oₓ.

Shaped unsupported catalyst bodies K7 were produced in the same way as shaped unsupported catalyst body K5, with a modification in f).

The granular material obtained was compacted (tableted) in f) with the aid of a Korsch PH 865 rotary press (with 65 dies) under a dry air atmosphere to give ringlike shaped precursor bodies of geometry E×H×I=5 mm×5 mm×2 mm with an uncurved (i.e. planar) end face of mass (Mᵥ) 166 mg. The speed of the rotor was 40 rpm. The side crushing strength (SDFᵥ) of the resulting ringlike shaped precursor bodies was 10 N.

### Example 8

Production of shaped unsupported catalyst body K8, where the active multimetal oxide had the stoichiometry Mo₁₂Co_{8.3}Fe_{2.1}Bi_{0.6}Si_{1.6}K_{0.08}Oₓ.

Shaped unsupported catalyst bodies K8 were produced in the same way as shaped unsupported catalyst body K7, with a modification in f).

The shaped precursor bodies were compacted (tableted) to a mass (Mᵥ) of 185 mg. The side crushing strength (SDFᵥ) of the resulting ringlike shaped precursor bodies was 16 N.

### Example 9 (not According to the Invention)

Production of shaped unsupported catalyst body K9, where the active multimetal oxide had the stoichiometry Mo₁₂Co_{8.3}Fe_{2.1}Bi_{0.6}Si_{1.6}K_{0.08}Oₓ.

Shaped unsupported catalyst bodies K9 were produced in the same way as shaped unsupported catalyst body K7, with a modification in f).

The shaped precursor bodies were compacted (tableted) to a mass (Mᵥ) of 205 mg. The side crushing strength (SDFᵥ) of the resulting ringlike shaped precursor bodies was 26 N.

### Example 10 (not According to the Invention)

Production of the shaped unsupported catalyst body K10, where the active multi metal oxide had the stoichiometry [Bi₂W₂O₉ 2WO₃]_{0.40}×[Mo₁₂Co_{5.4}Fe_{3.1}Si_{1.5}K_{0.08}Oₓ]₁.

The shaped unsupported catalyst bodies K10 were produced according to example I of WO 2010/000720.

The ringlike shaped precursor bodies of geometry E×H×I=5 mm×5 mm×2 mm with an uncurved (i.e. planar) end face had a mass (My) of 129 mg.

Testing of the annular unsupported catalysts K1 to K10 produced for a heterogeneously catalyzed partial oxidation of propene to acrolein and acrylic acid:

For the shaped unsupported catalyst bodies K1 to K6, a reaction tube (type 1.4541 stainless steel (EU standard number EN 10088-3); external diameter 33.7 mm; wall thickness 2 mm; internal diameter 29.7 mm; length 400 cm, 4 mm thermowell) was used.

For the shaped unsupported catalyst bodies K7 to K10, a reaction tube (type 1.4541 stainless steel (EU standard number EN 10088-3); external diameter 30 mm; wall thickness 2 mm; internal diameter 26 mm; length 400 cm, no thermowell) was used.

The respective reaction tube was charged as follows from the bottom upward:
Section 1: length 60 cm
   preliminary bed of steatite rings of geometry 7 mm×3 mm×4 mm (external diameter×length×internal diameter; C220 steatite from CeramTec);
Reaction zone: consisting of 2 sections (see table 3; zone 1 is the closest to the reactor inlet)
Section 2: length 30 cm
   downstream bed of the same steatite rings as in section 1;
Section 3: empty tube

Table 3 shows the structure of the reaction zone. Relative volume-specific catalyst activity was adjusted by diluting shaped unsupported catalyst bodies with inert steatite rings of geometry 5 mm×3 mm×2 mm (external diameter×length×internal diameter, C220 steatite from CeramTec) or of geometry 5 mm×5 mm×2 mm (external diameter×length×internal diameter, C220 steatite from CeramTec).

A reaction gas mixture conducted through the respective reaction tube charged as described above, flowing through the reaction tube from the top downward, had the following contents:
5.8% to 6.4 vol % of propene,
1.5% to 3 vol % of H₂O,
0.2% to 0.5 vol % of CO,
0.6% to 1.0 vol % of CO₂,
0.02% to 0.05 vol % of acrolein,
10.2% to 11.7 vol % of O₂, and
as the remainder to 100% molecular nitrogen.

Over the length of the reaction tube, a stirred and externally electrically heated salt bath (mixture of 53% by weight of potassium nitrate, 40% by weight of sodium nitrite and 7% by weight of sodium nitrate; 50 kg of salt melt) flowed around the reaction tube in each case (the flow rate at the tube was 3 m³/h (in the plane at right angles to the longitudinal axis of the tube)).

The temperature in the catalyst bed of the unsupported catalysts K1 to K6 was measured continuously by a thermocouple that was positioned in a thermowell in the interior of the reactor tube and was pushed from the bottom upwards in the reactor bed with the aid of a pulling device. The maximum temperature of this measurement corresponded to the hotspot temperature TH.

The drop test is conducted in this document as follows:

50 g of shaped unsupported catalyst bodies is allowed to fall through a vertical tube of length 3.5 m and clear diameter 23 mm. The shaped unsupported catalyst body falls into a porcelain dish immediately below the tube and is separated from dust and material broken off on impact. The intact shaped unsupported catalyst bodies that have been separated from dust and broken-off material are weighed. The proportion of broken shaped unsupported catalyst bodies is determined by comparison of the mass determined here with the mass of the shaped unsupported catalyst bodies used. The proportion of broken shaped unsupported catalyst bodies is a measure of the mechanical stability of the shaped unsupported catalyst bodies.

The selectivity of product of value formation (S (mol %)) in this document means:

$$S = \frac{\begin{matrix}number of moles of propene converted \\ to acrolein and acrylic acid \times 100\end{matrix}}{number of moles of propene converted overall}$$

(the conversion numbers are each based on a single pass of the reaction gas mixture through the fixed catalyst bed).

Comparison of the respective pairs of examples E1/E2, E3/E4 and E5/E6 and comparison of examples E7 and E8 with examples E9 and E10 shows a much higher product of value selectivity and much higher product of value yield (with lower catalyst mass) for the shaped unsupported catalyst bodies of the invention. At the same time, the shaped unsupported catalyst bodies of the invention have adequate mechanical stability.

TABLE 1
Shaped precursor bodies/shaped unsupported catalyst bodies

| | Shaped precursor body | | | | | Shaped unsupported catalyst body | | | | | | | |
|---|---|---|---|---|---|---|---|---|---|---|---|---|---|
| | Geometry | | | | MoO₃ | Bi₂O₃ | Fe₂O₃ | CoO | Weight loss | Geometry | | | |
| | E × H × I | Weight | Density | SDFᵥ | [% by | [% by | [% by | [% by | [% by | E × H × I | Weight | Density | SDFᵥ |
| Example | [mm] | [mg] | [g/ml] | [N] | wt.] | wt.] | wt.] | wt.] | wt.] | [mm] | [mg] | [g/ml] | [N] |
| K1 | 5 × 5 × 2 | 188 | 2.28 | 23 | 62.0 | 5.0 | 8.6 | 19 | 34.0 | 4.90 × 4.90 × 1.90 | 124 | 1.57 | 10 |
| K2*) | 5 × 5 × 2 | 220 | 2.64 | 48 | 62.0 | 5.0 | 8.6 | 19 | 34.1 | 4.90 × 4.90 × 1.90 | 145 | 1.83 | 15 |
| K3 | 5 × 3 × 2 | 90 | 1.80 | 6 | 62.0 | 5.0 | 8.6 | 19 | 33.9 | 4.84 × 2.95 × 1.87 | 60 | 1.29 | 4 |
| K4*) | 5 × 3 × 2 | 122 | 2.44 | 22 | 62.0 | 5.0 | 8.6 | 19 | 32.8 | 4.86 × 2.98 × 1.85 | 82 | 1.73 | 9 |
| K5 | 5 × 3 × 2 | 102 | 2.04 | 12 | 61.4 | 5.0 | 6.0 | 22 | 31.4 | 4.87 × 2.91 × 1.86 | 70 | 1.51 | 7 |
| K6*) | 5 × 3 × 2 | 119 | 2.38 | 26 | 61.4 | 5.0 | 6.0 | 22 | 31.1 | 4.85 × 2.97 × 1.84 | 82 | 1.75 | 7 |
| K7 | 5 × 5 × 2 | 166 | 1.99 | 10 | 61.4 | 5.0 | 6.0 | 22 | 33.1 | 4.83 × 4.90 × 1.84 | 111 | 1.45 | 7 |
| K8 | 5 × 5 × 2 | 185 | 2.22 | 16 | 61.4 | 5.0 | 6.0 | 22 | 33.0 | 4.85 × 4.92 × 1.85 | 124 | 1.60 | 10 |
| K9*) | 5 × 5 × 2 | 205 | 2.46 | 26 | 61.4 | 5.0 | 6.0 | 22 | 32.7 | 4.86 × 4.96 × 1.83 | 138 | 1.75 | 12 |
| K10*) | 5 × 3 × 2 | 129 | 2.61 | 21 | 57.0 | 6.2 | 8.2 | 13 | 27.5 | 4.90 × 2.95 × 1.90 | 94 | 1.96 | 10 |

*)comparative example
Weight loss = weight loss on thermal treatment
SDFᵥ = side crushing strength

TABLE 2
Shaped unsupported catalyst body

| | Drop test | BET | PV | PV (0.01-0.1) | PV (0.1-1) | PV (1-10) | PV (0-300) |
|---|---|---|---|---|---|---|---|
| Example | [% by wt.] | [m²/g] | [ml/g] | [%] | [%] | [%] | [%] |
| K1 | 6.0 | 6.0 | 0.37 | 2.8 | 93.4 | 0.4 | 3.5 |
| K2*) | 8.0 | 6.4 | 0.27 | 1.3 | 93.4 | 2.1 | 3.1 |
| K3 | 8.3 | 7.9 | 0.48 | 3.1 | 95.0 | 0.4 | 1.5 |
| K4*) | 9.7 | 6.4 | 0.32 | 4.4 | 92.3 | 0.4 | 2.9 |
| K5 | 7.0 | 7.0 | 0.39 | 7.9 | 89.1 | 0.5 | 2.4 |
| K6*) | 7.0 | 6.9 | 0.29 | 11.7 | 85.2 | 0.4 | 2.7 |
| K7 | 5.5 | 6.4 | 0.38 | 2.2 | 92.5 | 3.1 | 2.2 |
| K8 | 2.6 | 6.3 | 0.34 | 2.7 | 94.5 | 0.4 | 2.4 |
| K9*) | 2.9 | 6.3 | 0.30 | 3.3 | 93.7 | 0.5 | 2.5 |
| K10*) | 6.0 | 7.0 | 0.28 | 15.4 | 82.0 | 0.4 | 2.2 |

*)comparative example
BET = BET surface area
PV = total pore volume of pores having a diameter of > 0.03 to < 300 μm
PV (0.01-0.1) = volumetric proportion of pores having a diameter of > 0.01 to < 0.1 μm in the total pore volume
PV (0.1-1) = volumetric proportion of pores having a diameter of > 0.1 to < 1 μm in the total pore volume
PV (1-10) = volumetric proportion of pores having a diameter of > 1 to < 10 μm in the total pore volume
PV (10-300): = volumetric proportion of pores having a diameter of > 10 to < 300 μm in the total pore volume

TABLE 3
Filling of the reaction tubes

| | Length of | | Size of | | Dilution with | | Mass of | | Mass of |
|---|---|---|---|---|---|---|---|---|---|
| | reaction | | steatite rings | | steatite rings | | overall | | catalyst |
| | zone [cm] | | E × H × I [mm] | | [% by wt.] | | charge [g] | | charge [g] |
| Example | Zone 1 | Zone 2 | Zone 1 | Zone 2 | Zone 1 | Zone 2 | Zone 1 | Zone 2 | Zone 1 + 2 |
| E1 | 100 | 200 | 5 × 5 × 2 | — | 35 | 0 | 635 | 1115 | 1528 |
| E2*) | 90 | 170 | 5 × 5 × 2 | — | 35 | 0 | 633 | 1111 | 1522 |
| E3 | 100 | 180 | 5 × 3 × 2 | — | 45 | 0 | 510 | 731 | 1012 |
| E4*) | 100 | 180 | 5 × 3 × 2 | — | 40 | 0 | 592 | 874 | 1229 |
| E5 | 100 | 180 | 5 × 3 × 2 | — | 40 | 0 | 555 | 845 | 1178 |
| E6*) | 100 | 180 | 5 × 3 × 2 | — | 40 | 0 | 630 | 982 | 1360 |
| E7 | 100 | 180 | 5 × 5 × 2 | — | 40 | 0 | 489 | 723 | 1016 |
| E8 | 100 | 180 | 5 × 5 × 2 | — | 40 | 0 | 528 | 828 | 1145 |
| E9*) | 100 | 180 | 5 × 5 × 2 | — | 40 | 0 | 543 | 861 | 1187 |
| E10*) | 100 | 180 | 5 × 3 × 2 | — | 30 | 0 | 550 | 926 | 1311 |

*)comparative example

TABLE 4
Reaction conditions/yield

| | Propene space | Inlet | Salt bath | Hotspot | Propene | | |
|---|---|---|---|---|---|---|---|
| | velocity | pressure | temperature | temperature | conversion | Selectivity | Yield |
| Example | [I (STP)/I h] | [bar] | [°C] | [°C] | [%] | [mol %] | [mol %] |
| E1 | 125 | 1.0 | 333 | 414 | 97.1 | 94.3 | 91.6 |
| E2*) | 125 | 1.0 | 337 | 399 | 96.8 | 93.5 | 90.5 |
| E3 | 130 | 1.3 | 348 | 400 | 96.5 | 94.6 | 91.3 |
| E4*) | 130 | 1.3 | 337 | 401 | 96.5 | 93.7 | 90.4 |
| E5 | 130 | 1.3 | 337 | 396 | 96.5 | 94.5 | 91.2 |
| E6*) | 130 | 1.3 | 337 | 395 | 96.5 | 94.1 | 90.8 |
| E7 | 130 | 1.3 | 328 | — | 96.5 | 94.4 | 91.1 |
| E8 | 130 | 1.3 | 328 | — | 96.5 | 94.1 | 90.8 |
| E9*) | 130 | 1.3 | 329 | — | 96.5 | 93.8 | 90.5 |
| E10*) | 130 | 1.5 | 331 | — | 96.4 | 93.5 | 90.1 |

*)comparative example
Propene space velocity = quotient of propene mass flow rate (I (STP)/h) and empty volume of the reaction zone (I)
Selectivity = product of value selectivity (acrolein + acrylic acid)
Yield = product of value yield (acrolein + acrylic acid)

1.-15. (canceled)

16. A process for producing shaped unsupported catalyst bodies for gas phase oxidation of an alkene or an alcohol to an α,β-unsaturated aldehyde or an α,β-unsaturated carboxylic acid, wherein the shaped unsupported catalyst bodies comprise at least the elements molybdenum, bismuth, iron and cobalt, the process comprising:
a) producing an aqueous solution or aqueous suspension comprising at least one source of the elemental constituents molybdenum, bismuth, iron, cobalt and optionally at least one source of the elemental constituent nickel,
b) drying the aqueous solution or aqueous suspension obtained in a) to produce a powder P and optionally comminuting,
c) compacting the powder P obtained in b) to shaped precursor bodies having a cylindrical structure, optionally with addition of one or more auxiliaries and after uniform mixing and optional compaction, and
d) subjecting the shaped precursor bodies obtained in c) to thermal treatment to form the shaped unsupported catalyst bodies,
wherein the pressure in the compaction in c) is chosen such that the density of the shaped precursor bodies is from 1.70 to 2.30 g/cm³, where the density of the shaped precursor bodies is the quotient of the mass and geometric volume, and the weight loss in the thermal treatment in d) is from 25% to 40% by weight.

17. The process according to claim 16, wherein the cylindrical structure is
i) a cylindrical body having a central circular aperture in the longitudinal direction or
ii) a cylindrical body having three uniform notches in the longitudinal direction and having three circular apertures arranged uniformly in the longitudinal direction.

18. The process according to claim 16, wherein the molybdenum content of the shaped unsupported catalyst bodies, calculated as MoO₃, is from 45% to 75% by weight.

19. The process according to claim 16, wherein the bismuth content of the shaped unsupported catalyst bodies, calculated as Bi₂O₃, is from 1% to 20% by weight.

20. The process according to claim 16, wherein the iron content of the shaped unsupported catalyst bodies, calculated as Fe₂O₃, is from 2% to 12% by weight.

21. The process according to claim 16, wherein the total cobalt and nickel content of the shaped unsupported catalyst bodies, calculated as CoO and NiO, is from 9% to 30% by weight.

22. A shaped unsupported catalyst body having a cylindrical structure for gas phase oxidation of an alkene or an alcohol to an α,β-unsaturated aldehyde or an α,β-unsaturated carboxylic acid, obtained by a process of claim 16, wherein the shaped unsupported catalyst body comprises at least the elements molybdenum, bismuth, iron and cobalt, wherein the density of the shaped unsupported catalyst body is from 1.20 to 1.70 g/cm³, wherein the density of the shaped unsupported catalyst body is the quotient of the mass and geometric volume, the total pore volume of the shaped unsupported catalyst body is from 0.33 to 0.60 cm³/g and the pore volume of the shaped unsupported catalyst body in the range from 0.1 to 1 μm is from 85% to 99% of the total pore volume, and wherein the total pore volume and the pore volume in the range from 0.1 to 1 μm is determined by mercury porosimetry.

23. The shaped unsupported catalyst body according to claim 22, wherein the cylindrical structure is
i) a cylindrical body having a central circular aperture in the longitudinal direction or
ii) a cylindrical body having three uniform notches in the longitudinal direction and having three circular apertures arranged uniformly in the longitudinal direction.

24. The shaped unsupported catalyst body according to claim 23, wherein the shortest distance between the outer wall of the cylindrical body and the next aperture is from 0.75 to 2.5 mm.

25. The shaped unsupported catalyst body according to claim 22, wherein the density of the shaped unsupported catalyst body is from 1.35 to 1.55 g/cm³.

26. The shaped unsupported catalyst body according to claim 22, wherein the total pore volume of the shaped unsupported catalyst body is from 0.36 to 0.45 g/cm³.

27. The shaped unsupported catalyst body according to claim 22, wherein the pore volume of the shaped unsupported catalyst body in the range from 0.1 to 1 μm is from 88% to 96% of the total pore volume.

28. A process for preparing an α,β-unsaturated aldehyde or an α,β-unsaturated carboxylic acid, wherein an alkene or an alcohol is guided together with molecular oxygen over a fixed catalyst bed comprising a bed of shaped unsupported catalyst bodies according to claim 22.

29. The process according to claim 28, wherein propene is used as the alkene.

30. A fixed bed reactor comprising a bed of shaped unsupported catalyst bodies according to claim 22.
